(19) United States
(12) Patent Application Publication
(10) Pub. No.: US 2019/0038778 A1
(43) Pub. Date: Feb. 7, 2019

(54) Hybrid System for Efficient Gene Delivery to Cells of the Inner Ear

(71) Applicants: The General Hospital Corporation, Boston, MA (US); President and Fellow of Harvard College, Cambridge, MA (US)

(72) Inventors: Casey A. Maguire, Arlington, MA (US); David P. Corey, Newton, MA (US); Bence Gyorgy, Stoneham, MA (US)

(21) Appl. No.: 16/075,537

(22) PCT Filed: Feb. 3, 2017
(86) PCT No.: PCT/US17/16566

§ 371 Date: Aug. 3, 2018

Related U.S. Application Data
(60) Provisional application No. 62/291,871, filed on Feb. 5, 2016.

(51) Int. Cl. A61K 48/00; A61K 9/00

(52) U.S. Cl. CPC A61K 48/0075; A61K 9/0046; A61K 48/0058

(57) ABSTRACT

Methods for introducing a gene into a cell of the inner ear, e.g., a cochlear or vestibular cell, e.g., a hair cell, e.g., for therapy, that include the use of exosomes associated with one or more adeno-associated viral (AAV) particles.

### CLAIM OF PRIORITY

This application claims the benefit of U.S. Provisional Application Ser. No. 62/291,871, filed on Feb. 5, 2016. The entire contents of the foregoing are incorporated herein by reference.

### FEDERALLY SPONSORED RESEARCH OR DEVELOPMENT

This invention was made with Government support under Grant Nos. R01-DC002281 and R21 NS081374-01 awarded by the National Institutes of Health. The Government has certain rights in the invention.

### TECHNICAL FIELD

Described herein are methods for introducing a gene into a cell of the inner ear, e.g., a cochlear or vestibular hair cell, e.g., for therapy, that include the use of exosomes associated with one or more adeno-associated viral (AAV) particles.

### BACKGROUND

Hearing loss, congenital or acquired (most commonly age-related hearing loss), is a major health issue which affects approximately 30 million people in the United States alone 1, compared to about 5.4 million for Alzheimer's Disease 2. Congenital hearing loss has an incidence of about 1:1,000 births 3, of which about half have a defined genetic cause. Because the cochlea is surgically accessible and local application into a relatively immune-protected environment is possible, gene therapy using viral vectors is an attractive approach for treating hearing loss. For congenital recessive deafness, gene addition is possible, while congenital dominant forms might be treated by silencing or correcting the mutated gene 4. Gene therapy also holds promise for age-related hearing loss by targeting pathways involved in hair cell or spiral ganglion neuron survival (e.g. neurotrophic factors 5 or antioxidant proteins 6, 7), or by manipulating gene expression in supporting cells to induce their transdifferentiation into hair cells 8. For congenital hereditary hearing loss, at least 70 genes are causative in humans. In many cases they affect the function of hair cells, the receptor cells of the inner ear.

### SUMMARY

Adeno-associated virus (AAV) is a safe and effective vector for gene therapy for retinal disorders. Gene therapy for hearing disorders is not as advanced, in part because gene delivery to sensory hair cells of the inner ear is inefficient. Although AAV transduces the inner hair cells of the mouse cochlea, outer hair cells remain refractory to transduction. Here, we demonstrate that a vector, exosome-associated AAV (exo-AAV), is a potent carrier of transgenes to all inner ear hair cells. Exo-AAV1-GFP is more efficient than conventional AAV1-GFP, both in mouse cochlear explants in vitro and with direct cochlear injection in vivo. Exo-AAV shows no toxicity in vivo, as assayed by tests of auditory and vestibular function. Finally, exo-AAV1 gene therapy partially rescued hearing in a mouse model of hereditary deafness (Lhfpl5/Tmhs^{-/-}). Exo-AAV is a powerful gene delivery system for hair cell research and may be useful for gene therapy for deafness. As used herein, the term “exosome” encompasses all extracellular vesicles including microparticles and microvesicles.

Thus, provided herein are methods for inducing expression of a transgene in a cell of the inner ear. The methods include delivering to the cell an effective amount of an exosome-associated viral vector comprising the transgene.

In some embodiments, the cell is a hair cell of the cochlea or vestibular system. In some embodiments, the cell is an inner hair cell of the cochlea or an outer hair cell of the cochlea; in some of these embodiments, the subject has a hearing disorder, and the transgene is delivered in a therapeutically effective amount.

In some embodiments, the cell is a cell of the vestibular system, e.g., a hair cell of the utricle, or a cell in an ampulla of a lateral semicircular canal, or a hair cell in a cupula. In some embodiments wherein the cell is a cell of the vestibular system, the subject has a disorder of the vestibular system, and the transgene is delivered in a therapeutically effective amount.

In some embodiments, the transgene is listed in Table A.

In some embodiments, the exosomes are 50-150 nM in diameter.

Also provided herein are compositions comprising an exosome-associated viral vector comprising a transgene for use in inducing expression of the transgene in a cell of an inner ear of a subject. In some embodiments, the transgene is listed in Table A.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-E. Exo-AAV outperforms conventional AAV in hair cell transduction in culture. (a) Standard (conventional) AAV and exo-AAV production workflow. AAV was purified from HEK (human embryonic kidney)-293T cell lysate, while exo-AAV was isolated from the culture medium of the cells. Cryo-electron microscopy shows AAV1 capsids associated with exosomes. White arrowheads show AAV capsids, while black arrowhead indicates the lipid membrane. Scale bars are 50 nm. (b) Transduction of cochlear whole mount cultures with AAV1 or exo-AAV1. Cochleas were explanted from CD1 mice at P1. Vectors were added (10¹¹ genomic copies [GCs]) the following day and incubated overnight. Organs were cultured for three more days. Exo-AAV1-GFP shows efficient transduction of inner hair cells (IHC) and outer hair cells (OHC); hair cells were labeled with anti-myosin VIIa antibody. Scale bar is 20 μm. (c) Proportion of GFP-positive hair cells in cochleas transduced with 1×10¹¹ GC of conventional AAV1 or exo-AAV1. Numbers in the bars represent the number of cochleas. Three images were taken for each cochlea (base, middle, apex; fields chosen by distance). Mean±SEM, ***p<0.001, **p<0.01, *p<0.05, one-tailed t-test. (d) Proportion of GFP-positive hair cells in different regions of the cochlea (basal, middle and apical turns) transduced with conventional AAV1 or exo-AAV1. n=6 cochleas for each data point, **p<0.01, one-tailed t-test. (e) GFP-positive hair cells in cochleas transduced with 1×10¹¹ GC of conventional AAV9 or exo-AAV9. Mean±SEM, ***p<0.001, *p<0.05, one-tailed t-test.

FIGS. 2A-D. Exo-AAV outperforms conventional AAV in hair cell transduction in vivo. CD1 mice were injected at postnatal day 1 with 5×10⁹ GC of conventional AAV1 or exo-AAV1 either by cochleostomy or through the round window membrane. (a) Efficient OHC transduction by exo-AAV1 compared to conventional AAV1. The insets in the lower panels show the outlined region of the main panel at the same magnification but with higher brightness. All image post-processing was done identically between AAV1 and exo-AAV1. Scale bars are 20 μm. (b) Proportion of GFP-positive hair cells in cochleas transduced with conventional AAV1 vs. exo-AAV1. Numbers in the boxes represent the number of injected animals. Eight images were acquired for each sample and four were analyzed. Mean±SEM, ***p<0.001, **p<0.01, *p<0.05, one-tailed t-test. The experiment was carried out on separate occasions, using three different litters for each vector and different exo-AAV preparations, with 38 mice/cochleas total. For RWM injection, we performed the injections on two separate litters for each vector (23 mice/cochleas total). Bars represent the combined results from all animals. (c) GFP fluorescence intensity in GFP-positive hair cells. Hair cells were identified by myosin VIIa-fluorescence-based segmentation (Imaris); n=6 cochleas for cochleostomy in each group and n=4 for RWM injection in each group. Four images were analyzed per cochlea. GFP negative (GFP level below +2 SD background) cells were excluded from the intensity analysis. (d) Percentage of GFP-positive hair cells in four regions of the cochlea (base, midbase, midapex, apex), transduced with conventional AAV1 or exo-AAV1. Mean±SEM) ***p<0.001, **p<0.01, *p<0.05, Mann Whitney U test between AAV1 and exo-AAV1. R² is the coefficient of determination for the average values in each region; it tests whether there is a correlation between the location and transduction efficiency. Most conditions showed more transduction in the base for cochleostomy but not RWM injection. Repeated measures ANOVA test assuming equal sphericity ***p<0.001, **p<0.01, *p<0.05.

FIGS. 3A-C. Exo-AAV1 transduces utricular hair cells after RWM injection. CD1 mice were injected with 5×10⁹ GC of conventional AAV1-CBA-GFP or exo-AAV1-CBA-GFP. (a) GFP indicates all transduced cells; myosin VIIa labels just hair cells. Scale bar is 40 μm. (b) Higher magnification shows many transduced cells identified also express myosin VIIa (white arrows). Scale bar is 20 m (c) Blinded unbiased quantification of transduced hair cells. p<0.05, Mann Whitney U test. Numbers in the boxes indicate the number of biological replicates.

FIGS. 4A-D. exo-AAV1-HA-Lhfpl5 rescues FM1-43 loading in hair cells in culture. Lhfpl5^{+/-} or Lhfpl5^{-/-} cochleas (C57BL/6 background) were dissected at P0 and placed into culture for 8 days. exo-AAV1-HA-Lhfpl5 was added to the culture at P0. At P8, Tmc2 is no longer expressed and so is no longer an alternate path for FM1-43 loading. (a) FM1-43 loading indicating functional hair cells in control Lhfpl5^{+/-} mice. Knockout Lhfpl5^{-/-} animals showed no loading, but loading was evident in the Lhfpl5^{-/-} animals after vector administration (2×10¹¹ GC). Scale bar is 20 μm. (b) LHFPL5 in stereociliary bundles of KO mice after vector-mediated Lhfpl5 gene delivery, revealed with anti-HA staining. Hair bundle actin was labeled with phalloidin (red). (c) FM1-43 signal intensity measured with ImageJ. Het: Lhfpl5^{+/-}, KO: Lhfpl5^{-/-}, GC: genomic copes. Exo-AAV1-CBA-HA-Lhfpl5 administration led to increased FM1-43 signal intensity. ***p<0.001, t-test. (d) FM1-43 signal intensity in Lhfpl5^{+/-}, Lhfpl5^{-/-} and exo-AAV1-HA-Lhfpl5-rescued Lhfpl5^{-/-} animals (2×10¹¹ GC) in different regions of the cochlea. *p<0.05, t-test.

FIGS. 5A-D. RWM injection of exo-AAV1-HA-Lhfpl5 induces LHFPL5 bundle expression in hair cells and rescues FM1-43 loading. (a) HA-LHFPL5 detected with immunolabeling for the HA tag. Cochleas from Lhfpl5^{-/-} mice (C57BL/6 background) were injected through the round window at P1 with exo-AAV1-CBA-HA-Lhfpl5. Hair bundle actin was stained with phalloidin (red). HA staining is apparent in the IHC and OHC bundles, as well as some supporting cells. P4+2 days in culture. Scale bar is 20 μm. (b) High magnification images show anti-HA staining in the bundles of an inner and an outer hair cell. Anti-HA staining is detectable at the tips of all rows of stereocilia. Scale bar is 2 μm. (c) RWM injection of exo-AAV1-CBA-HA-Lhfpl5 through the round window at P1 restores FM1-43 loading in IHCs and OHCs (7 days after injection; P6+2). Scale bar is 20 μm. (d) Regional transduction efficiency based on HA staining in bundles of apical, middle and basal regions of the cochlea (P4+2) (n=4). No difference was apparent between different regions.

FIGS. 6A-D. RWM injection of exo-AAV1-HA-Lhfpl5 improves hearing and improves movement abnormalities in Lhfpl5^{-/-} animals. (a) Auditory brainstem response (ABR) waveforms at 8 kHz from heterozygous, uninjected Lhfpl5^{-/-} and exo-AAV1-CBA-HA-Lhfpl5 injected Lhfpl5^{-/-} animals. Sound pressure level is shown in decibels (dB). ABR was recorded at 4 weeks post-injection. (b) ABR thresholds (mean±SD). Left, heterozygous control mice injected with exo-AAV1-CBA-HA-Lhfpl5 through round window membrane injection. Thresholds were not changed by exo-AAV injection. Right, Lhfpl5^{-/-} knockout mice injected with exo-AAV1-CBA-HA-Lhfpl5 through the RWM at P1. Solid black circles represent uninjected Lhfpl5^{-/-} ears, and show no detectable ABR at any sound pressure level. Open circles show heterozygous control thresholds at this age (data from left panel non-injected). Colored symbols represent nine individual animals that showed some level of rescue after exo-AAV1-CBA-HA-Lhfpl5 injection. (c) Peak 1 (P1) and peak 2 (P2) ABR wave amplitudes and latencies at 8 and 11 kHz. ABR peaks were normal in both uninjected and injected control heterozygotes, but were never detected in uninjected knockout animals. ABR peaks were smaller but present in exo-AAV1-injected knockouts. Waveforms were measured at 4 weeks post injection. p<0.01, t-test. (d) Behavior tests to monitor movement abnormalities in treated and untreated mice. Left, head tossing was quantified by blinded investigators. Head tossing in exo-AAV1-CBA-HA-Lhfpl5 injected knockouts was less than in uninjected knockouts, and not detected at all in 5 out of 12 injected animals (green circles). Right, circling was also decreased in exo-AAV1-CBA-HA-Lhfpl5 injected knockouts. Circling was quantified using EthoVision XT software and full 360 degree turns were identified as a circle. *p<0.05, ***p<0.001, Mann Whitney U test.

FIGS. 7A-D. AAV1 associates with exosomes. (a,b) Cryo-electron microscopy images showing AAV on the surface (a) or the interior (b) of exosomes. Black arrowheads point to exosomal membrane; white arrowheads show AAV capsids on the exterior of the vesicle; white arrows show capsids that appear to be on the interior of the exosome; and black arrow shows one AAV capsid distorting the membrane suggesting it is on the interior of the vesicle. Scale bars are 50 nm. (c, d) Transmission electron micrographs of cryosectioned exo-AAV samples that were immunogold-labeled to detect intact AAV1 capsids. AAV capsids are seen on the surface (white arrowheads, c and d) or inside exosomes (white arrow, d). Black arrowheads indicate exosome membrane. Scale bars are 50 nm (c) or 100 nm (d).

FIGS. 8A-B. Exo-AAV9-CBA-GFP outperforms conventional AAV9-CBA-GFP in transduction of cochlear explant hair cells. (a) In vitro. Cochleas were explanted from CD1 mice at P1. Vectors were added at 1×10¹¹ GC the next day for overnight transduction and organs were cultured for three more days. Exo-AAV9-GFP efficiently transduces IHCs and OHCs, labeled with anti-myosin VIIa antibody. Viral transduction is quantified in FIG. 1e. Scale bar is 20 μm. (b) In vivo. Exo-AAV9 was injected by cochleostomy. The number of hair cells transduced with exo-AAV9 in vivo was similar that transduced with exo-AAV1. Scale bar is 30 μm. Right, there was no difference between exo-AAV1 and exo-AAV9 in vivo. Exo-AAV1 values are replotted from FIG. 2b for reference. Mean±SEM.

FIGS. 9A-L. Transduction of cochlear cells by exo-AAV1-GFP and AAV1-GFP vectors administered by cochleostomy. Middle turn of a cochlea at low magnification. CD1 mice were injected at P1, then cochleas were dissected at P14. Hair cells were stained with anti-myosin VIIa antibody (red). (a-f) Exo-AAV1. Panels a,d,e,f are confocal images at four different depths. (g-l) AAV1. Panels g,j,k,l are confocal images at four different depths. For both, other cell types are also efficiently transduced. OC: organ of Corti, IHC: inner hair cell, OHC: outer hair cell, SG: spiral ganglia neurons, ISC: inner sulcus cells, HC: Hensen cells, CC: Claudius cells, NF: nerve fibers, F: fibroblasts. Scale bars: 60 μm.

FIGS. 10A-B. Transduction of the vestibular sensory epithelium by exo-AAV1-GFP and AAV1-GFP vectors, administered by cochleostomy. (a) Low magnification. CD1 mice were injected at P0/P1; the utricle and the ampulla of the lateral semicircular canal were dissected at P14. Hair cells were stained with antibodies to myosin VIIa (purple); actin was labeled with phalloidin (red); and GFP is green. Many vestibular hair cells and supporting cells were transduced, even though the vector was delivered to the cochlea. Scale bars are 30 μm. (b) High magnification. Images show colocalization of GFP with myosin VIIa in hair cells (arrows). Scale bars are 10 μm.

FIG. 11. Comparison of exo-AAV1-GFP transduction efficiency in CD1 and C57BL/6 mice; injection by cochleostomy. The difference between CD1 (data from FIG. 2b) and C57BL/6 was not significant for either IHCs or OHCs (Mann-Whitney U test). Mean±SEM.

FIG. 12. Maximum observed transduction using exo-AAV1 delivered by cochleostomy. C57BL/6 mice were injected by cochleostomy using 0.3 μl of exo-AAV1-CBA-GFP at P1 (resulting in 6×10⁹ GC injected). Cochleas were dissected at P14. Images represent the cochlea with the highest number of GFP-positive hair cells (assessed with direct GFP fluorescence). Scale bar is 20 μm.

FIGS. 13A-C. Schematic of the vectors used in the study. (a) Self-complimentary (sc) AAV-Lhfpl5 constructs. We synthesized a mouse codon-optimized AAV expression cassette containing a hemagglutinin (HA) tag on the N-terminus. Expression was driven by the chicken beta-actin (CBA) promoter. (b) Western blot of protein from AAV-producer 293T cells using anti-HA. LHFPL5 protein has a molecular weight of 24 kDa. (c) Schematic of the single stranded (ss) AAV-CBA-HA-Lhfpl5-IRES-GFP construct. IRES: internal ribosomal entry site.

FIG. 14. Co-expression of GFP and HA-LHFPL5 after in vivo injection of exo-AAV-CBA-HA-Lhfpl5-IRES-GFP. Lhfpl5^{-/-} mice (C57BL/6 background) were injected through the round window at P1 and cochleas were dissected at P6. The single-stranded construct transduced IHCs and OHCs, revealed by GFP expression. Furthermore, GFP-positive cells showed anti-HA labeling in the cell body and in the bundle, confirming the correlation between GFP expression and LHFPL5 localization in bundles. Scale bars are 400 μm (left panel) and 20 μm (right panel).

### DETAILED DESCRIPTION

The cochlea has two types of hair cells: Inner hair cells (IHCs) convert the mechanical stimulus of sound vibration into a neural signal transmitted by type I spiral ganglion neurons to the brain. Outer hair cells (OHCs) connect only to poorly-defined type II neurons; their main function is to amplify the vibration produced by sound by as much as 60 decibels (dB) in a frequency-specific manner, and they are essential for frequency discrimination⁹ (important in speech perception). Most deafness genes known to affect hair cell function are expressed in both cell types, so in general, a useful gene therapy strategy should target both IHCs and OHCs. Cells, including hair cells, in the vestibular system, e.g., in the semicircular ducts (horizontal, anterior and posterior) or two otolith organs (saccule and utricle), are essential for our sense of balance and for coordinating eye movements; they are often affected in hereditary deafness so gene therapies should target them as well.

The major limitation of gene therapy for the cochlea or vestibular system is the relative inefficiency of vectors that mediate transgene expression in hair cells. Several gene delivery strategies have targeted sensory cells in the cochlea, including viral and non-viral methods (see^{10, 11} for review). As yet, however, none of these has led to efficient transgene expression in hair cells. AAV vectors are presently the most promising vectors for cochlear gene delivery, but in vivo transduction is mostly limited to IHCs. In previous studies^{12, 13}, virtually no OHCs were transduced by AAV vectors after in vivo injection in mice. Similarly, it is difficult to express genes in hair cells in vitro for research, which has slowed characterization of proteins involved in hair cell function. There is therefore a great need for a vector system that effectively transduces both IHCs and OHCs, both in vitro and in vivo. It would pave the way to clinical trials, and would also be useful in studying hair cell physiology.

In the present disclosure, exosome-associated AAV vectors (exo-AAVs) were tested for delivery to cochlear hair cells and compared different injection routes to the cochlea in mice. Exosomes are cell-derived natural lipid structures involved in intercellular communication and are potential therapeutic carriers of nucleic acids and proteins (see ¹⁴ and ¹⁵). While exosome association of AAV enhances transduction of other types of cells in vitro and in vivo^{16, 17}, it had not been shown that exosomes would also augment gene delivery into transduction-resistant cochlear or utricular hair cells.

As shown herein, exosome-associated AAV transduces cochlear and vestibular hair cells with much greater efficiency than do conventional AAV vectors. Prior studies had shown some transduction of IHCs with conventional AAVs, but little transduction of OHCs^{12, 23}. The present disclosure shows that exo-AAV1 efficiently transduces cells of the inner ear including IHCs, and transduces OHCs much more efficiently than does conventional AAV1. AAV1 was the standard of comparison as it has been used in prior studies of hearing gene therapy^{12, 13} and in those studies was shown to transduce only inner hair cells relatively efficiently. Interestingly, exo-AAV9-GFP was extremely efficient at transduction of cochlear explant cultures, although in vivo this serotype performed similarly to exo-AAV1.

In order to determine whether the efficient reporter gene levels in HCs achieved with exo-AAV could be translated to expression of HC-relevant genes, exo-AAV mediated expression of Lhfpl5 was tested in deaf Lhfpl5^{-/-} mice. After injection through the round window membrane (RWM) into the scala tympani, there was widespread expression of HA-tagged LHFPL5 throughout the cochlea. Treated Lhfpl5^{-/-} mice were able to respond to sound, as measured physiologically, and showed improvements in balance-related abnormal movement, assessed behaviorally.

The present methods achieved hearing thresholds that were 20 dB better than in a recent rescue of Tmc1 deficiency with conventional AAV1¹³. OHC transduction was also confirmed by immunostaining for the HA tag and FM1-43 loading. Interestingly, the best rescue was in the 8-11 kHz range, as also found by Askew et al.¹³. Vector transduction efficiency was relatively even along the cochlea, so this frequency-dependent rescue could be because mouse hearing is normally most sensitive in that range.

In a similar study, Akil et al. used a conventional AAV vector to achieve robust hearing rescue in VGLUT3 KO mice¹². However, the VGLUT3 KO only affects inner hair cells, not outer hair cells, and so is easily rescued by conventional AAV1. Indeed, the authors showed that virtually no outer hair cells were transduced with conventional AAV1. In contrast, exo-AAV allows efficient transduction of outer hair cells as well, and is thus applicable to the great majority of deafness genes that are required for function in both inner and outer hair cells. In gene therapies where both hair cell types require correction, conventional AAV1 will likely not suffice.

The published behavioral phenotype of Tmhs (Lhfpl5) mutant mice is circling and abnormal head movements such as shaking and tossing, which are indicative of a balance disorder²⁴. We observed that exo-AAV1-HA-Lhfpl5 treated Lhfpl5 KO mice had improvements in both circling and in head tossing behavior. We also observed robust transduction of hair cells in the vestibular system in these mice. Together this may suggest a rescue of balance dysfunction in these mice, although further testing using measurements such as vestibular evoked potentials (VsEPs) could be done to confirm.

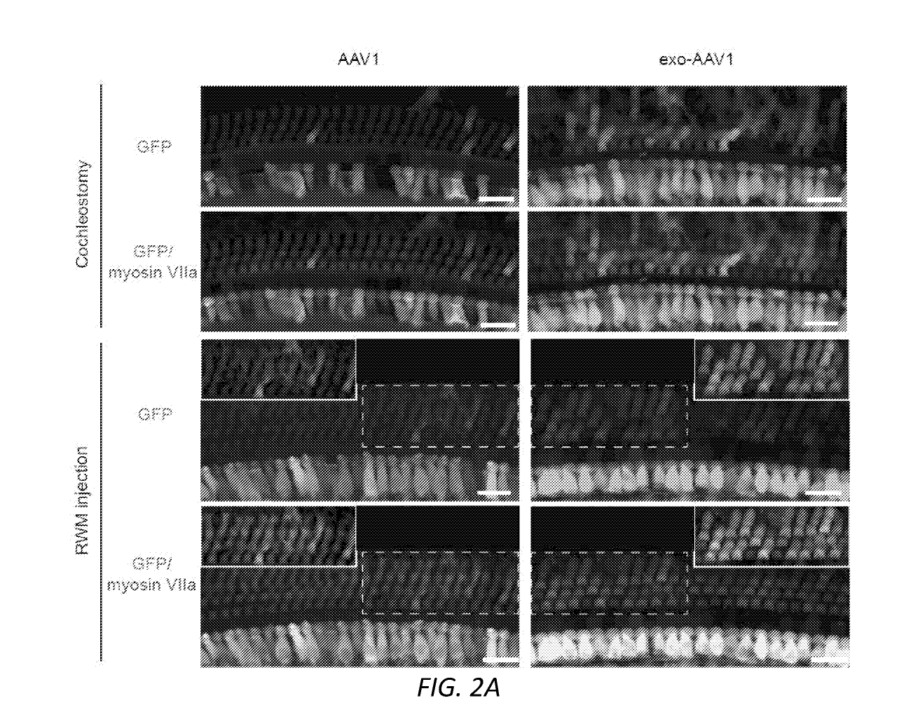

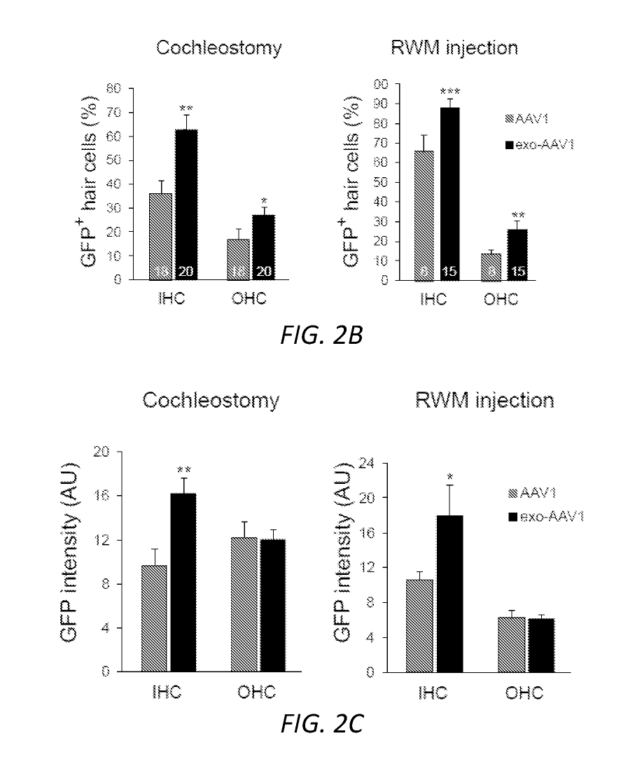

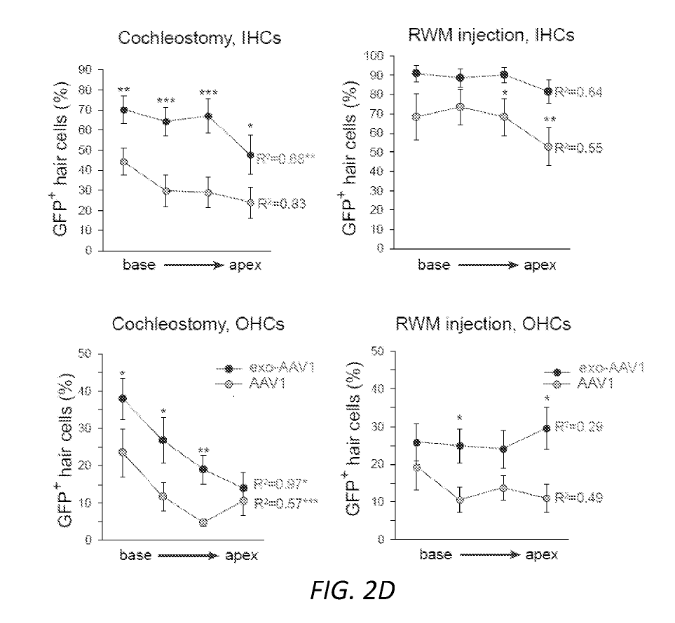

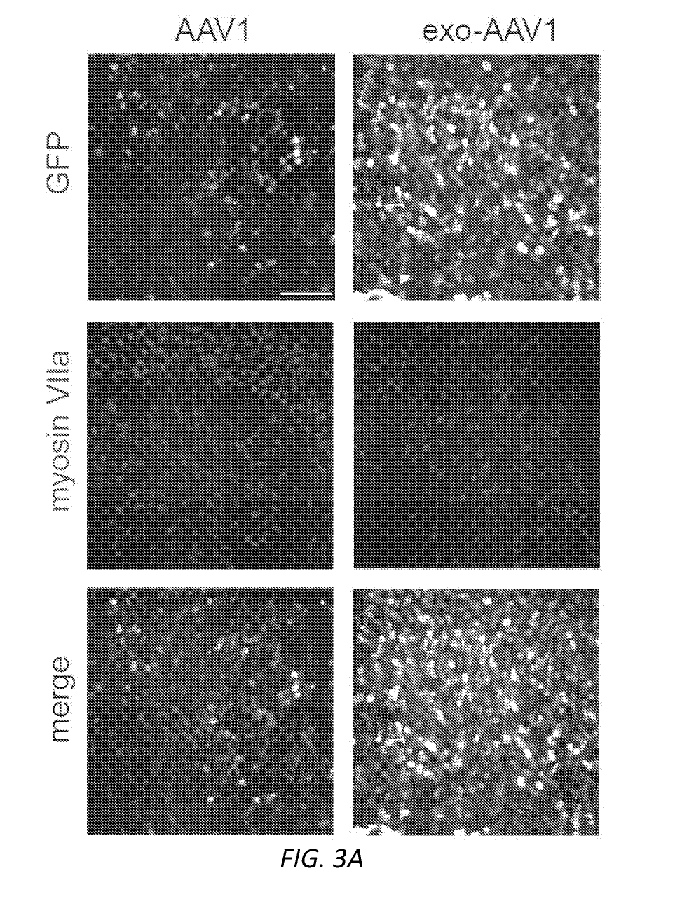

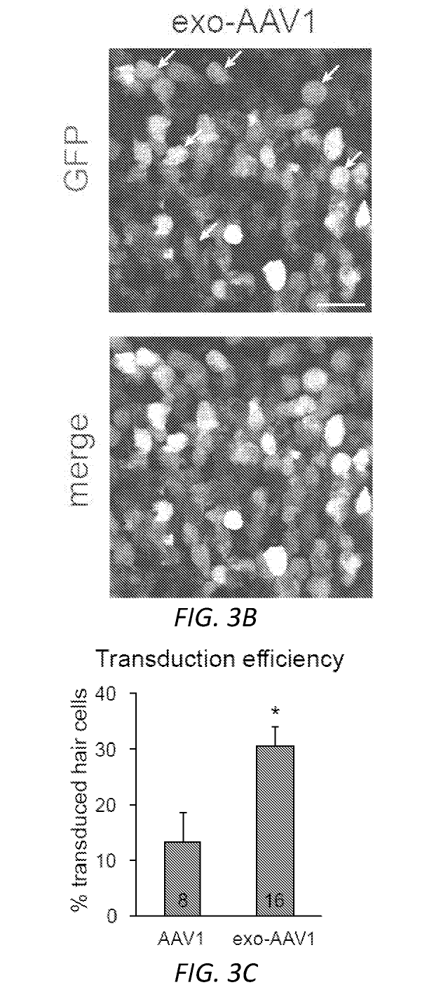

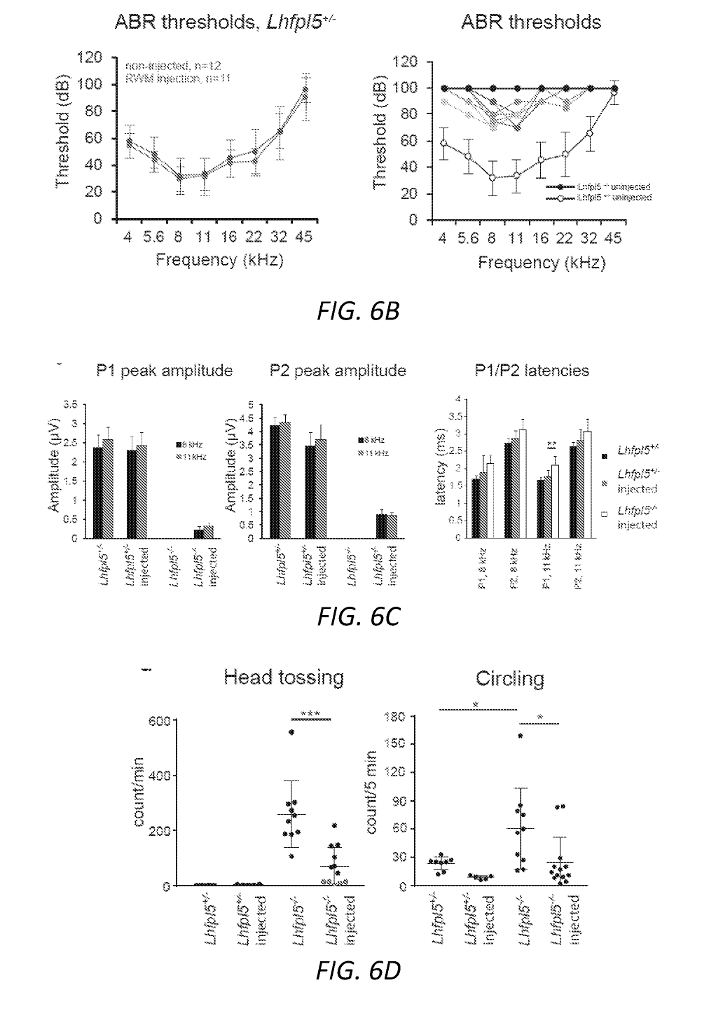

We used self-complementary (sc) AAV vector genomes in our transduction comparisons between exo-AAV and AAV (FIGS. 2 and 3) as well as for rescue experiments (FIG. 6). Sc genomes are genetically engineered variants of the natural single stranded (ss) AAV genome^{25, 26} that give rise to complementary, half-sized AAV genomes that fold into a double-stranded-like structure. Sc genomes are thought to bypass the second-strand synthesis step required for transcription of transgenes from ssAAV vectors. Bypassing this step generally leads to an earlier onset and more robust level of transgene expression, although it comes with the cost of reduced cassette size (˜2.4 kb compared to 4.7 kb for ss vectors). That said, many genes fit into sc vectors, including Lhfpl5, and a scAAV vector is in clinical trials for the treatment of spinal muscular atrophy (See, e.g., Choudhury et al., Neuropharmacology. 2016 Feb. 21. pii: S0028-3908(16)30048-X. doi: 10.1016/j.neuropharm.2016.02.013. [Epub ahead of print]; clinicaltrials.gov). It may be promising for certain deafness genes.

Exo-AAV is relatively easy to purify¹⁹, allowing rapid production of several different constructs for experimental testing. In contrast, purification of conventional AAV is a more complicated and time-consuming process. It is important to note, also, that if new AAV capsids with enhanced transduction in hair cells are discovered, they can be incorporated into the exo-AAV system for potentially even better performance.

Thus, exo-AAV vectors are efficient delivery vehicles for mammalian hair cells of the inner ear, both in vitro and in vivo. Exo-AAV-mediated genetic modification of inner and outer hair cells should facilitate elucidation of the basic biology of hair cells, and provides an avenue for gene therapy for human hereditary deafness.

### Exosome-Mediated Viral Gene Delivery to the Inner Ear

The methods and compositions described herein can incorporate a gene construct to be used as a part of a gene therapy protocol. The invention includes methods for using exosomes to enhance delivery of viral expression vectors for in vivo and in vitro transfection and expression of a polynucleotide that encodes a therapeutic polypeptide or active fragment thereof, or a protein that increases expression, level, or activity of a protein, in inner ear cells, especially cochlear or utricular hair cells.

Viral Vectors

Viral vectors for use in the present methods and compositions include recombinant retroviruses, adenovirus, adeno-associated virus, alphavirus, and lentivirus.

A preferred viral vector system useful for delivery of nucleic acids to the inner ear in the present methods is the adeno-associated virus (AAV). AAV is a tiny non-enveloped virus having a 25 nm capsid. No disease is known or has been shown to be associated with the wild type virus. AAV has a single-stranded DNA (ssDNA) genome. AAV has been shown to exhibit long-term episomal transgene expression, and AAV has demonstrated excellent transgene expression in the brain, particularly in neurons. Vectors containing as little as 300 base pairs of AAV can be packaged and can integrate. Space for exogenous DNA is limited to about 4.7 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics. For example, AAV9 has been shown to efficiently cross the blood-brain barrier. Moreover, the AAV capsid can be genetically engineered to increase transduction efficient and selectivity, e.g., biotinylated AAV vectors, directed molecular evolution, self-complementary AAV genomes and so on. Modified AAV have also been described, including AAV based on ancestral sequences; see, e.g., U.S. Pat. No. 7,906,111; WO/2005/033321; WO2008027084, WO2014124282; WO2015054653; and WO2007127264. In some embodiments, AAV1 is used. In some embodiments, AAV9 is not used.

Alternatively, retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 d1324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

Alphaviruses can also be used. Alphaviruses are enveloped single stranded RNA viruses that have a broad host range, and when used in gene therapy protocols alphaviruses can provide high-level transient gene expression. Exemplary alphaviruses include the Semliki Forest virus (SFV), Sindbis virus (SIN) and Venezuelan Equine Encephalitis (VEE) virus, all of which have been genetically engineered to provide efficient replication-deficient and -competent expression vectors. Alphaviruses exhibit significant neurotropism, and so are useful for CNS-related diseases. See, e.g., Lundstrom, Viruses. 2009 June; 1(1): 13-25; Lundstrom, Viruses. 2014 June; 6(6): 2392-2415; Lundstrom, Curr Gene Ther. 2001 May; 1(1):19-29; Rayner et al., Rev Med Virol. 2002 September-October; 12(5):279-96.

Exosome-Associated Viruses

The methods provided herein address some limitations of using viral vectors such as AAV vectors for gene transfer through the use of exosomes. The characteristics of exosomes are well suited for the compositions and methods provided herein. An exosome is small, nanometers in size, and can contain DNA, mRNA and microRNA (miRNA). Exosomes contain a lipid membrane and host proteins that are recognized as self by the immune system. Exosomes can encapsulate or otherwise package a wide variety of molecules, including nucleic acids and proteins. The exosomes used in the compositions and methods provided herein are useful for shielding the viral capsid from pre-existing neutralizing antibodies.

Methods for generating the exosome-associated viral vectors are described in US20130202559. Briefly, the media from viral producer cells is collected and exosomes containing viral vectors are purified. In the present methods, exosomes of 50-150 nm in diameter are preferentially isolated from media from the producer cells for use, e.g., using ultrafiltration or ultracentrifugation. This differs from the standard procedure for the production of viral vectors such as AAV. In the standard procedure for AAV production, the AAV vectors are produced by triple transfection of 293T cells with plasmids encoding for structural, nonstructural, and helper virus genes required for replication and virus production, and then, the virus is harvested and purified from cell lysates and the media is discarded. In the methods and studies described herein, the media from producer cells (i.e., cells that produce and shed exosomes) is harvested, rather than the cells. For example, the exosomes in the media can be pelleted, the pellet resuspended and then loaded onto a density gradient. The fractions are collected and analyzed.

In the studies provided herein, the natural ability of cells to secrete exosomes was exploited. Following transfection of 293T cells with an AAV2 vector construct and capsid expression cassettes in a standard vector production paradigm, it was discovered that a substantial fraction of AAV virons released from the cells were within exosomes, e.g., exo-AAV. Intact AAV capsids within individual exosomes were observed by transmission electron microscopy.

Suitable producer cells for use in the purified exosome populations, compositions and methods of the invention include, by way of non-limiting example, cells such as 293T (ATCC, see also Biotechniques. 2003 January; 34(1): 184-9); Per.C6 (Crucell), AGE1.CR (ProBioGen AG); AGE1.HN® cell line (ProBioGen AG); and KG-1 cells (ATCC, see also Biochem Biophys Res Commun. 2005 Aug. 5; 333(3):896-907), which can be differentiated into human dendritic cells. Suitable producer cells also include tumor cells such as cells from ovarian cancer, glioma, hepatocarcinoma (see e.g., PLoS One. 2010 Jul. 22; 5(7):e11469), as tumor-derived exosomes have been shown to have immunosuppressive properties and can be useful for long-term expression of a transgene. Suitable producer cells also include regulatory T cell lines such as CD4+CD25+ T cells (see e.g., Blood. 2005 Nov. 1; 106(9):3068-73. Epub 2005 Jul. 14), which can be useful for long-term expression of a transgene.

In some embodiments, the lipid membrane of the exosome is modified to enhance or otherwise alter a property of the exosome, such as, for example, target cell type, cell activation, or a transduction property. For example, the expression or presence of a cell surface protein found on the exosome can be altered to induce a change in the exosome. In some embodiments, the surface of the exosome can be modified to include a receptor ligand that targets a desired cell type or a bridging molecule linked to a receptor ligand that targets a desired cell type.

In clinical settings, the gene delivery systems described herein can be introduced into the inner ear of a subject by any of a number of methods, each of which is known in the art. For instance, a cochleostomy, injection through the round window, or injection into one of the semicircular canals can be used.

The pharmaceutical preparation of the gene therapy construct can consist essentially of the gene delivery system in an acceptable diluent, e.g., saline and/or physiologically acceptable media, and/or can comprise a slow release matrix in which the gene delivery vehicle is embedded.

Therapeutic Genes for Treating Hearing or Balance Disorders

The present methods can be used to deliver (or enhance delivery of) genes to cells, e.g., hair cells, of the cochlea or utricle. In some embodiments, the genes correct a genetic defect; see, e.g., Smith et al., Deafness and Hereditary Hearing Loss Overview. 1999 Feb. 14 [Updated 2014 Jan. 9]. In: Pagon et al., editors. GeneReviews® [Internet]. Seattle (Wash.): University of Washington, Seattle; 1993-2017. In other embodiments, the genes promote survival of hair cells after a toxic insult. Exemplary genes and the associated conditions are listed in Table A.

TABLE A

| Disease or | | NCBI RefSeq ID | | |
|---|---|---|---|---|
| condition | Gene | GENE | Protein | C/V |
| Autosomal | TMIE | NG_011628.1 | NP_671729.2 | C |
| Recessive | | | | |
| Nonsyndromic | | | | |
| Hearing Loss | | | | |
| Autosomal | TMC1 | NG_008213.1 | NP_619636.2 | C |
| Recessive | | | | |
| Nonsyndromic | | | | |
| Hearing Loss | | | | |
| Sensorineural | ATOH1 | NC_000004.12 | NP_005163.1 | C |
| hearing loss | | | | |
| Autosomal | Lhfp15 | NG_012184.1 | NP_872354.1 | CN |
| Recessive | | | | |
| Nonsyndromic | | | | |
| Hearing Loss | | | | |
| Usher syndrome | C1rn1 | NG_009168.1 | NP_777367.1 | C |
| type 3a | | | | |
| Usher syndrome | Ush1C | NG_011883.1 | NP_001284693.1 | CN |
| type 1c | (harmonin) | | | |
| Waardenburg | PAX3 | NG_011632.1 | NP_852122.1 | C |
| syndrome I, III | | | | |
| Waardenburg | MITF | NG_011631.1 | NP_000239.1 | C |
| syndrome II | | | | |
| Waardenburg | EDNRB | NG_011630.2 | NP_000106.1 | C |
| syndrome IV | EDN3 | NG_008050.1 | NP_996917.1 | |
| | SOX10 | NG_007948.1 | NP_008872.1 | |
| Pendred syndrome | SLC26A4 | NG_008489.1 | NP_000432.1 | C |
| | (Pendrin) | | | |
| Episodic Ataxia 1 | KCNA1 | NG_011815.1 | NP_000208.2 | V |
| Episodic Ataxia 2 | CACNA1A | NG_011569.1 | NP_000059.3 | V |
| Episodic Ataxia 5 | CACNB4 | NG_012641.1 | NP_001005747.1 | V |
| Episodic Ataxia 6 | SLC1A3 | NG_015890.1 | NP_004163.3 | V |
| Various forms of | CX26/GJB2 | NG_008358.1 | NP_003995.2 | C |
| autosomal | | | | |
| recessive | | | | |
| deafness | | | | |

C: cochlea-hearing disorder,
V: vestibular system disorder

Additional conditions and the associated genetic defects have been described in the art; see, e.g., Kemperman et al., J R Soc Med. 2002 April; 95(4): 171-177; Gazquez and Lopez-Escamez, Curr Genomics. 2011 September; 12(6): 443-450; Duan et al., Gene Therapy (2004) 11:S51-S56.

Thus, encompassed within the present disclosure is a composition comprising an exosome-associate virus, wherein the virus comprises a sequence as shown in Table A. Further, these compositions can be used to treat a condition associated with loss of hearing or vestibular dysfunction, wherein the condition is caused by a genetic defect or is ameliorated by genetic therapy, e.g., a condition listed in Table A. Thus, in some embodiments, the methods described herein are used to treat a condition listed in table A, using the corresponding sequence listing in Table A, in a subject in need thereof. Examples include certain forms of Usher syndrome (deafness associated with blindness and in some forms vestibular dysfunction).

Subjects who can be treated using the present methods include mammals, e.g., humans and non-human veterinary subjects including experimental animals. In some embodiments, the subjects have a condition listed in Table A. Subjects having these conditions can be identified using diagnostic methods known in the art.

In some embodiments, the viral vector also includes regulatory sequences, e.g., promoter sequences, for expression of the transgene in a cell of the inner ear. Suitable sequences can include the Atohl enhancer region and/or the Myo7a promoter/intron.

### EXAMPLES

The invention is further described in the following examples, which do not limit the scope of the invention described in the claims.

Materials and Methods

The following materials and methods were used in the present examples.

Animals.

All experiments were performed in compliance with ethical regulations and approved by the Animal Care Committee of Harvard Medical School. For in vitro and in vivo studies we used CD1 mice (Charles River), housed and bred in the animal facility at Harvard Medical School. Wild-type C57BL/6 animals were ordered from Charles River. Lhfpl5 heterozygous and homozygous knockout (KO) animals were housed and bred in our facility. Male and female mice were randomly chosen for study. Pilot experiments allowed us to estimate sample size for animal experiments.

Genotyping.

For genotyping we used the following primers: wild type exon 2 forward: TGACTGCTGGATCTCAGTGC (SEQ ID NO: 1), wild type exon 2 reverse: GTTTGGCTGCTGGTCTTAGC (SEQ ID NO:2), Lhfpl5 KO forward: TAGCAGGCATGCTGGGGATG (SEQ ID NO:3), Lfhpl5 KO reverse: TCCGCTGATGGCCTTTCTCA (SEQ ID NO:4). PCR conditions were as follows: 95° C. for 2 min followed by 9 cycles of 95° C. 30 sec, 66° C. 30 sec (−1° C./cycle), 72° C. 30 sec, then 25 cycles of 95° C. 30 sec, 57° C. 30 sec, 72° C. 30 sec, finally 75° C. for 5 minutes. The PCR products were separated on an agarose gel. Lhfpl5^{-/-} allele shows a 238 bp band, wild-type allele shows a 577 bp band.

Vector Preparation.

We isolated conventional AAV and exo-AAV vectors from transfected HEK-293T cells, as previously described^{16, 17}. For each production we plated two 15-cm tissue culture dishes with 1.5×10⁷ HEK-293T cells. The next day cells were transfected using the calcium phosphate method, with the adenovirus helper plasmid (pAdΔF6²⁷, 26 μg), rep/cap plasmid (pXR1²⁸ for AAV1, pAR9 for AAV9, 12 μg) and ITR-flanked transgene cassette plasmid (10 μg) to induce production of AAV. All plasmids were obtained from the Massachusetts General Hospital virus vector core. Plasmids were diluted in 780 μl with 2.5 mM HEPES and 2M calcium chloride and then added drop-wise into 780 μl 2× HEPES-buffered saline (280 mM NaCl, 50 mM HEPES, 1.5 mM Na₂HPO₄, pH 7.04) while vortexing in 15 ml tubes. The mixture was incubated at room temperature for 20 min before adding it to cells drop-wise. The day after transfection, medium was changed to DMEM containing 2% FBS. The following day medium was changed to DMEM containing 2% exosome-free FBS (made by overnight 100,000 g ultracentrifugation to deplete bovine exosomes). Exo-AAV vectors were isolated from the media three days after transfection using differential centrifugation as described before¹⁶. Cells were depleted at 300 g for 5 min and 1000 g for 10 min. Next, larger extracellular vesicles (apoptotic bodies, exosomes) were removed by a 20,000 g spin for 60 min. The supernatant from the 20,000 g spin was subjected to 100,000 g centrifugation using a Type 70 Ti rotor in an Optima L-90K ultracentrifuge for 1.5 h (both Beckman Coulter, Ind., USA). The exosome pellet was re-suspended in serum-free, antibiotic-free DMEM medium. Conventional AAVs were purified from the cell lysate using iodixanol-gradient ultracentrifugation. Vectors were stored at −80° C. until use. For titration of exo-AAV vectors, we first treated the titration aliquot with DNase to remove plasmid DNA. Next, we isolated all capsid-protected nucleic acids from the sample using the Roche High Pure Nucleic Acid viral kit (Roche, Pleasanton, Calif., USA), in order to remove PCR inhibitors and nucleases potentially present in exosome preparations and to fully lyse the exosomal membrane. For titration of conventional AAV we have observed that the DNase I treatment and downstream nucleic acid purification steps are not required as the crude cell lysates are treated with Benzonase nuclease to remove plasmid DNA. Titration of standard AAV by simply diluting the sample before titration or using the DNase I/Roche High Pure Nucleic Acid viral kit procedure, as for exo-AAV, yielded identical titers. Finally, we quantified AAV genomic copies in conventional and exo-AAV preparations using TaqMan qPCR with BGH polyA-sequence specific primers and probe¹⁷. Titers for all vectors (AAV and exo-AAV) were in the 2×10¹²-1.5×10¹³ GC/ml range.

AAV Vector Constructs.

AAV transgene plasmid (AAV2 inverted terminal repeat (ITR)-flanked) encoding green fluorescent protein (GFP) under the hybrid CMV immediate-early/chicken beta actin (CBA) promoter, AAV-CBA-GFP, was kindly provided by Dr. Miguel Sena-Esteves (UMass Medical Center). AAV-CBA-GFP is a self-complementary (sc) genome. pAdΔF6²⁷, pXR1²⁸ (AAV1), AAV9 plasmids were all obtained from the Massachusetts General Hospital virus vector core. We constructed two AAV vectors encoding murine Lfhpl5. The transgene was designed from the coding region of mRNA of Mus musculus lipoma HMGIC fusion partner-like 5 (Lhfpl5) (NCBI Reference Sequence: NM_026571.2). A mouse codon-optimized version of mouse Lhfpl5 with an N-terminal human influenza hemagglutinin (HA) tag was synthesized and inserted into a cloning vector (pUC57-Kan) by Genscript (Piscataway, N.J.). To construct this plasmid, we first digested sc-AAV-CBA-GFP with HindIII and NheI to remove the GFP transgene. The pUC57-Kan plasmid was similarly digested to release codon-optimized, HA-tagged, Lhfpl5. This insert was ligated with the scAAV backbone to create scAAV-CBA-HA-Lhfpl5. A second construct was made using an ss AAV plasmid as backbone. For this construct we used the plasmid ssAAV-CBA-IRES-GFP kindly provided by Dr. Miguel Sena-Esteves. This plasmid contains a multiple cloning site after the CBA promoter and an internal ribosomal entry site (IRES)-driven GFP, allowing for co-expression of GFP and a gene of interest. We digested ssAAV-CBA-IRES-GFP with SpeI and NheI and dephosphorylated the plasmid with calf inositol phosphatase before agarose gel purification. We PCR-amplified HA-Lhfpl5 from scAAV-CBA-HA-Lhfpl5 using forward and reverse primers flanked with SpeI and NheI recognition sequences, respectively. The amplified product was digested overnight with SpeI and NheI and ligated with similarly digested ssAAV-CBA-IRES-GFP. After restriction digest screening for correct ligation orientation, ssAAV-CBA-HA-Lhfpl5-IRES-GFP was generated. Confirmational DNA sequencing was performed.

Transmission Electron Microscopy (TEM).

Exo-AAV vectors were pelleted and fixed for 30 minutes in 4% formaldehyde in PBS. The pellet was cryoprotected in 2.3M sucrose in PBS before it was frozen in liquid nitrogen. Cryosections (approximately 80 nm thick) were incubated with 1:100 dilutions of mouse anti-AAV1 antibody, which recognizes intact capsids (Clone ADK1a; American Research Products, Waltham, Mass., USA), followed by a 10-nm-gold conjugated secondary anti-mouse antibody (Sigma-Aldrich, St. Louis, Mo., USA). Images were acquired with a Tecnai G² Spirit BioTWIN transmission electron microscope (FEI Company, Hillsboro, Oreg., USA) in the Harvard Medical School Electron Microscopy Facility.

Cryo Electron Microscopy (CryoEM).

CryoEM was performed on conventional and exo-AAV1 vectors. Briefly, 4 μl of sample was deposited on electron microscopy grids coated with a perforated carbon film. After draining the excess liquid with a filter paper, grids were quickly plunged into liquid ethane and mounted onto a Gatan 626 cryoholder (Gatan, Pleasanton, Calif., USA). CryoEM observation was performed with a Tecnai F20 (Fei Company) microscope operated at 200 kV and images recorded with a USC1000-SSCCD camera (Gatan).

Immunoblotting.

293T cells were transfected for AAV production using scAAV-CBA-HA-Lhfpl5 as described above. Five days after transfection, cells were lysed using M-PER (mammalian protein extraction reagent, Thermo Scientific). Cell lysates were subjected to SDS PAGE gel electrophoresis and subsequently proteins transferred to a nitrocellulose membrane. For detection of the HA-tagged LHFPL5, we used a biotinylated anti-HA antibody (Biolegend, catalog number 901505) at 1:2,000 dilution followed by streptavidin conjugated to horseradish peroxidase (GE Healthcare, 1:25,000 dilution). Specific antibody binding was detected by incubating the membrane with Pierce ECL Western Blotting Substrate (Thermo Scientitic) and exposing autoradiographic film to the membrane.

Cochlear Culture.

To assess viral transduction by different vectors in vitro, we explanted cochleas from CD1 wild-type mice at postnatal day 1 (P1). Briefly, after dissecting out the temporal bone, we opened the bone and the cochlear coil from the scala vestibuli side. Next, we removed the stria vascularis and detached the coil from the modiolus. The spiral ligament was kept in place to improve plating of the cochleas. The specimen was plated onto a glass-bottom dish (P35G-1.5-14-C; Mattek, Ashland, Mass., USA) using a tissue glue (Cell-Tak, Corning, Corning, N.Y., USA). Cochleas were cultured in DMEM supplemented with 5% fetal bovine serum, 1% N2 supplement (Thermo-Fischer, Woburn, Mass., US) and 5 μg/ml carbenicillin. After overnight culture, we added the vector solution in 200 μl of medium at a dose of 10¹¹ GC per cochlea (unless indicated otherwise). After overnight incubation we changed the media and kept the cochleas in culture for 3 more days, to the equivalent of P6.

Cochlear Immunostaining and Imaging.

Cochleas were fixed with 4% formaldehyde in PBS for 20 minutes. Fixed cochleas were washed 3 times with PBS to remove fixative and were blocked with 5% normal goat serum and permeabilized with 0.3% Triton X-100 in PBS for 1 hour at 22° C. Primary antibodies were diluted in 5% NGS/0.1% Triton X-100/PBS and incubated overnight at 4° C. To stain hair cells, we used rabbit polyclonal anti-myosinVIIa antibody (Proteus Biosciences Inc., Ramona, Calif., USA, 1:500 dilution) with a goat anti-rabbit IgG secondary antibody conjugated to Alexa Fluor 647 in a 1:1,000 dilution for 1 hour (Life Technologies). To stain the hair bundle actin we used phalloidin conjugated to Alexa Fluor 544 (Life Technologies) (1:100). To stain for the HA tag, we used an anti-HA antibody (C29F4, rabbit; Cell Signaling Technology Inc., Danvers, Mass.). GFP was detected with its intrinsic fluorescence. Tissues were mounted on a Colorfrost glass slide (Thermo-Fischer Scientific, Waltham, Mass.) using Prolong Gold Antifade mounting medium (Thermo-Fischer). Imaging was performed with an Olympus FluoView 1000 confocal microscope (Olympus, Center Valley, Pa., USA) using a PlanApoN 60×/1.42NA oil-immersion objective.

FM1-43 Loading

We assessed the ability of hair cells to accumulate the styryl dye FM1-43, to test functional restoration of hair cell mechanotransduction in culture. Cultured cochleas were quickly washed twice with Leibovitz's L15 medium (Gibco) and incubated in the presence of 2 μM FM1-43 dye (Thermo-Fisher) for 30 seconds. Excess dye was removed by chelation using 0.2 mM SCAS (4-sulfonato calix[8]arene, sodium salt, from Biotium, Hayward, Calif., USA) for two minutes. Imaging was performed with an upright Olympus FluoView 1000 confocal microscope (Olympus, Center Valley, Pa., USA) using a LUMPLanFI/IR 60×/1.1 water-dipping objective.

In Vivo Cochlear Injections.

For in vivo analysis, we performed procedures on P0 or P1 wild-type mouse pups of the CD1 strain. After injection, we waited 14 days before sacrifice to assess vector transduction.

Cochleostomy.

P0-P1 CD1 pups were anesthetized by hypothermia, then kept on an ice pack during the procedure. A small incision was made underneath the external ear. The incision was enlarged and soft tissues were pushed apart using an eyelid retractor to expose the bulla. The bulla was then opened, following the stapedial artery, using a 25 m flattened microprobe. Soft connective tissues were removed to get access to the lateral wall of the cochlea. A glass pipet was inserted through the lateral wall perpendicular to the stria vascularis to a depth of 300 μm, then 250 nl of solution (containing 5×10⁹ GCs of AAV) was injected at a constant rate of 45 nl/min using a Nanoliter 2000 Injector (World Precision Instruments, Sarasota, Fla., USA). We closed surgical incisions with 2-3 sutures using a 7-0 Vycril surgical suture. Following surgery, the pups were maintained at 37° C. until complete recovery (10-15 minutes).

Round Window Membrane Injection.

As for cochleostomy, the bulla was exposed and opened. Then, the round window niche was localized visually. Covering connective tissues were removed in order to expose the round window. We injected 250 nl vector solution at rate of 45 nl/min for adequate comparison with cochleostomy (introducing 5×10⁹ GCs of AAV for GFP expression experiments). For rescue experiments, we injected 1-1.2 μl of the exo-AAV1-HA-Lhfpl5 construct into the ear (2.7×10⁹ GCs). We closed surgical incisions with 2-3 sutures using a 7-0 Vycril surgical suture.

Image Analysis.

To compare transduction efficiency between conventional AAV and exo-AAV, we determined the percentage of GFP-positive hair cells and the intensity of GFP expression. To determine the percent of GFP-positive cells, we manually counted GFP-positive and -negative cells in in vitro cultures for three regions in each cochlea (approximately 120 hair cells/image). For in vivo injections, we captured 8 high magnification images along the cochlea, at exactly the same distance from the base for all cochleas. Then in each case, an investigator blinded to the vector used counted all visible hair cells on 4 of the images (using always the locations) and determined the percentage of GFP-positive cells. For intensity determination we used Imaris 8.1 software (Bitplane AG, Zurich, Switzerland). Briefly, we segmented individual hair cells based on myosin VIIa fluorescence and quantified GFP fluorescence in IHCs and OHCs separately. Measurements were normalized to the GFP intensity values from hair cells in non-injected cochleas, segmented the same way. We only counted GFP-positive hair cells (scored as having a GFP intensity two standard deviations above mean background) to avoid transduction rate as a confounding variable. To quantify FM1-43 fluorescence intensity on z-stacks of images, we used ImageJ 1.46r software. When comparing different groups, we always used the same imaging settings, including zoom settings, pixel ratio and z-step size.

Auditory Brainstem Response (ABR).

The ABR assay was performed using a Tucker Davis Technologies workstation (System III; TDT, Gainsville, Fla.). Mice were anesthetized by intraperitoneal injection of a ketamine (100 mg/kg)/xylazine (10 mg/kg) cocktail. Anesthetized mice were then placed on a heating pad and electrodes were placed subcutaneously in the vertex, underneath the left or right ear, and on the back near the tail. Tone stimuli of 4, 5.6, 8, 11.2, 16, 22, 32 and 45.3 kHz were calibrated with a precision microphone system (PS9200 Kit; ACO Pacific, USA) using the TDT SigCal software package. The recorded signals were band-pass filtered (300 Hz to 3 kHz) and amplified 100,000 times. The number of acquisition trials was set to 500 averages. Maximum stimulus intensity was set to 95 dB peak SPL with attenuation decreasing from 85 dB to 0 dB SPL at 5 dB intervals. Bandpass filters (500-3,000 Hz) filters were applied to the traces before analysis.

Behavioral Tests Startle Response.

A simple startle response was performed. Briefly, animals were placed in an opaque white bucket and were allowed to equilibrate for several minutes in quiet. An investigator performed a hand clap which was not visible to the animals. The animals were filmed by a Panasonic HCV550 camera at the age of 4 weeks. Lhfpl5 knockout animals never responded.

Open Field Test and Behavioral Function Quantification.

We used a 37 cm diameter arena with low and even illumination. Animals were tested at 6 weeks age. Animals were placed on the side of the arena and were filmed for 5 minutes. After each mouse, the arena was cleaned to avoid olfactory distractions. To quantify head tossing and circling, videos were analyzed by two investigators who were blinded to the genotype or the injection status of the animals. For head tossing, 3 separate minutes were counted and averaged for each investigator. The average scores assigned by each separate investigator showed a strong positive correlation (Spearman r=0.86, p=1.76×10¹¹). Circling was quantified using the Ethovision XT software package (Noldus, Wageningen, The Netherlands). We counted full circle rotations during a 5 min observation period (360° turns in either clockwise or counterclockwise direction).

Head tossing and circling behavior were tested at two separate days. The results between the two days showed a strong correlation (Spearman test, for head tossing: rho=0.7, p<0.05, for circling: rho=0.85, p<0.001).

Statistics.

To compare two non-related sample groups, we used a t-test for two independent samples or a Mann-Whitney U test. For normality testing, we used the Shapiro-Wilk test. For correlations, we used Spearman correlation. To test the relationship between the location on the cochlea and virus transduction, we performed repeated measures ANOVA test. For statistical testing, we used the GraphPad Prism software (GraphPad Software, La Jolla, Calif., USA). p<0.05 were considered statistically significant.

### Example 1. AAV1 Associates with Exosomes, when Vectors are Isolated from Ultracentrifuged Cell Culture Medium

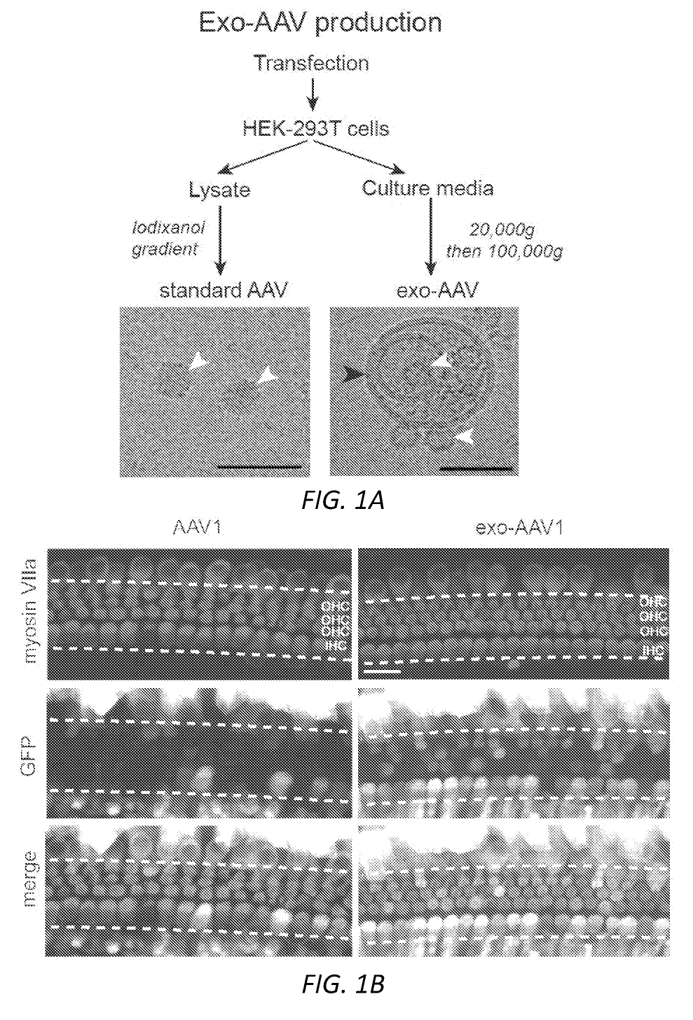

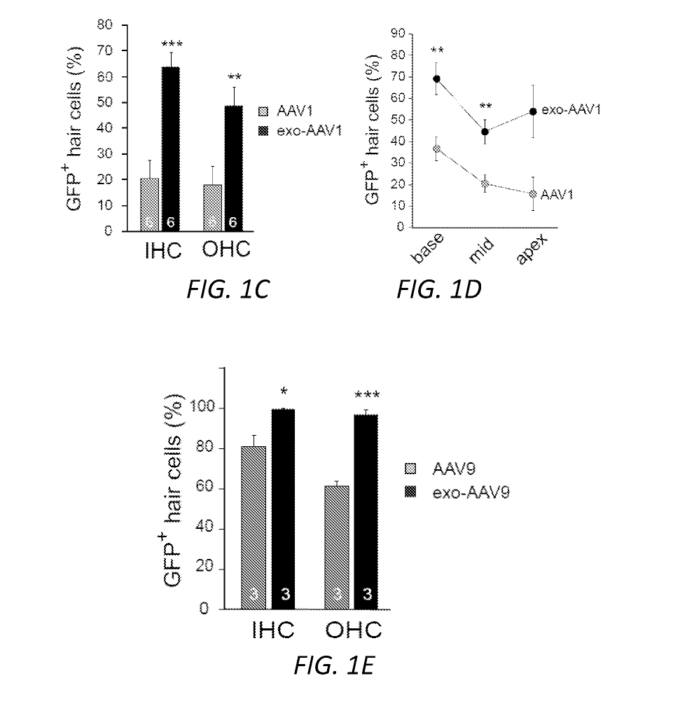

Of AAV serotypes, AAV1 (the number denotes the capsid serotype) has been reported to be the most effective for cochlear hair cell transduction in preclinical gene therapy studies^{12, 13}. We therefore prepared exo-AAV1 and conventional AAV1. AAV1 and exo-AAV1 were isolated from the lysate and culture medium of vector-producing 293T cells, respectively (FIG. 1a; see Methods). Using cryo-electron microscopy (EM) and transmission EM in combination with immunogold labeling, we qualitatively observed AAV capsids both bound to the surface of exosomes and in their interiors (FIG. 1a and Supplementary FIG. 1. a-d). The capsids lining the outer exosome membrane could be clearly distinguished as surface bound, while capsids that appear “inside” could be above, inside, or below the vesicle within the TEM section. However, we observed instances of capsids distorting the membrane from the interior of the vesicle, directly confirming that at least some of the capsids are indeed on the interior (Supplementary FIG. 1b).

### Example 2. Exo-AAV Outperforms Conventional AAV in Transgene Delivery to Cochlear Hair Cells in Explant Cultures

We first assessed transgene delivery efficiency of conventional AAV1 and exo-AAV1 vectors on cochlear explant cultures. Cochleas were dissected at postnatal day 1 (P1) and placed in organ culture. One day later, vectors were added to the culture medium. We used vectors encoding green fluorescent protein (GFP) under the strong hybrid CMV/chicken beta actin (CBA) promoter. After culturing the cochleas with the vectors for three days (equivalent to age P5), tissues were fixed, labeled with phalloidin and with antibodies to the hair cell marker myosin VIIa, and viewed with a confocal microscope.

Exo-AAV1 vector was superior to conventional AAV1 vector in gene delivery to hair cells (FIG. 1b-e). At 10¹¹ genomic copies (GC) per cochlea, conventional AAV1-GFP vector transduced approximately 20% of IHCs and OHCs, whereas exo-AAV1-GFP transduced up to 65% of IHCs and 50% of OHCs (p<0.001 and p<0.01, respectively; FIG. 1c). We also looked for regional differences in efficiency, and found exo-AAV1 outperformed conventional AAV1 at both middle and basal turns of the cochlea (FIG. 1d; p<0.01 for middle and basal turns, not significant for apical turn).

We also tested another serotype, AAV9, as both exo-AAV9 and conventional AAV9. At 10¹¹ GC/cochlea, we observed a significant enhancement of transduction by exo-AAV9 (FIG. 1e and Supplementary FIG. 2a). Strikingly, exo-AAV9 transduced almost 95% of IHCs and OHCs (FIG. 1e). We conclude that both exo-AAV1 and exo-AAV9 vectors significantly enhance both IHC and OHC transduction in cochlear explant cultures, compared to conventional AAV vectors.

### Example 3. Exo-AAV Outperforms Conventional AAV in Transgene Delivery to Cochlear Hair Cells In Vivo

These results indicate that exo-AAVs can be powerful gene-delivery vehicles for in vitro experimental work, but they may not reflect in vivo performance needed for therapy. We therefore compared conventional AAV1 and exo-AAV1 in vivo, using direct injections into P0/P1 mouse cochleas. We compared two injection routes: through the round window membrane (RWM) into the scala tympani (used in two animal models of hereditary deafness^{12, 13}), and through the lateral wall of the cochlea by cochleostomy at the basal turn. In both cases, 250 nl of virus-containing solution was injected over ˜10 min.

Conventional AAV1-GFP transduced some IHCs but few OHCs: cochleostomy delivery of conventional AAV1 vector resulted in only 36% GFP-positive IHCs and 17% GFP-positive OHCs (FIG. 2a, 2b). Upon RWM injection, we observed more GFP-positive IHCs (up to 65%) but fewer OHCs (˜14%) (FIG. 2a, 2b). With either route, however, exo-AAV1-GFP vectors significantly enhanced gene delivery: When delivered by cochleostomy, exo-AAV transduced 63% of IHCs and 28% of OHCs. With RWM injection, 88% of IHCs and 25% of OHCs were transduced (FIG. 2a, 2b).

As reported by Askew et al. ¹³, cochlear injection of P0 mouse pups is difficult, causing significant variability in transduction. Our results (FIG. 2b-d) include all of the injected mice (n=38 for cochleostomy; n=23 for RWM), even those with very low transduction efficiency that may result from unsuccessful injection. In our best injections with exo-AAV vectors, we observed >95% IHCs and ˜50% OHCs transduced with our working dose (250 nl, containing 5×10⁹ genomic copies of AAV). The best cases of the cochleostomy and RWM injections had similar hair cell transduction rates. However, in our hands, cochleostomy results were more variable and there were more instances with very low GFP expression.

Because GFP expression in individual hair cells may vary with multiple AAV genomes being delivered, we quantified GFP intensity using automated image analysis. Among GFP-positive IHCs, average GFP fluorescence intensity per cell was 70% higher with exo-AAV than with conventional AAV, with either cochleostomy or RWM injection (p<0.01 for cochleostomy, and p<0.05 for RWM injection; FIG. 2c). For OHCs, no significant difference in GFP intensity per cell was evident between exo-AAV1 and conventional AAV1.

For cochleostomy, transduction rates varied with distance from the injection site. We counted more transduced hair cells in the base (near the injection site) than in the apex (FIG. 2d). The gradient was particularly steep and significant for OHCs, with only a few OHCs transduced at the apex (repeated measures ANOVA for the entire dataset to analyze relationship between location and transduction, p=0.0009 for AAV1 and p=0.02 for exo-AAV1). With RWM injection, however, there was no significant gradient, suggesting that virus can diffuse more freely with this approach. Overall, in all subregions tested, with both injection routes, exo-AAV1 significantly outperformed conventional AAV1 (FIG. 2d).

With cochleostomy injections of either conventional or exo-AAV1, we also observed robust expression of GFP in spiral ganglion neurons, cells in the inner sulcus, Claudius cells, and Hensen cells (Supplementary FIG. 3).

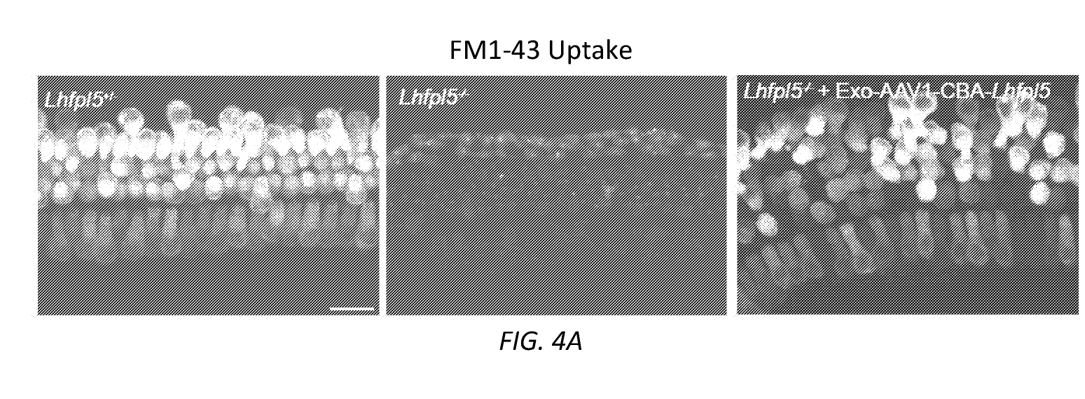

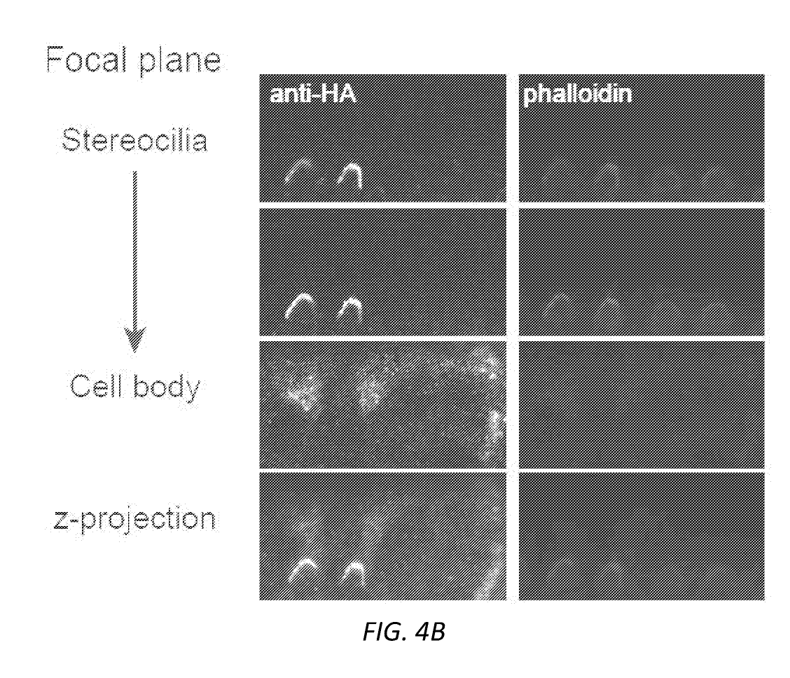

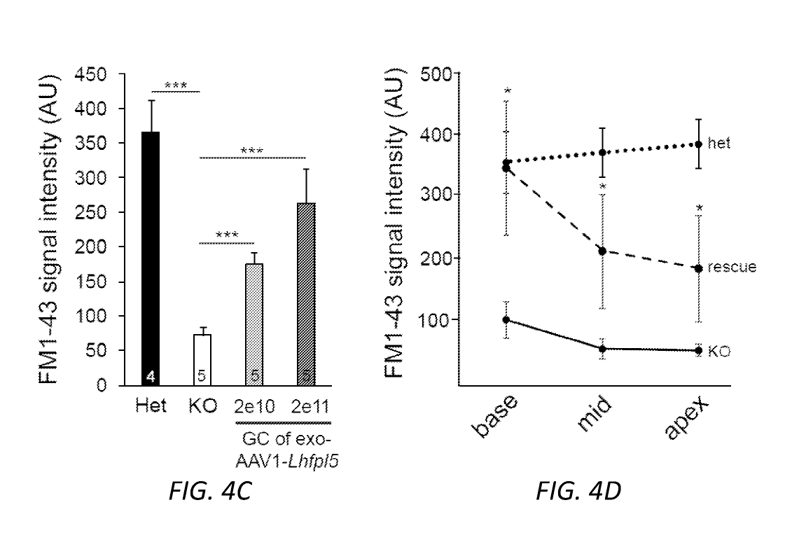

Surprisingly, GFP-positive hair cells were also evident in the utricle and in the ampullas of the lateral semicircular canals following exo-AAV administration by either cochleostomy (Supplementary FIG. 4) or RWM injection (FIG. 3a-b). In the utricle after RWM injection, exo-AAV1 transduced 30% of hair cells (FIG. 3c), 2.3 times more than conventional AAV1 (p<0.05, Mann Whitney U test), indicating some level of diffusion throughout endolymphatic compartments at that age. Several myosin VIIa-negative cells (supporting cells) were also transduced with either vector. Thus exo-AAV vectors also have potential for gene delivery to the vestibular system.

Because exo-AAV9 outperformed exo-AAV1 in vitro, we also tested AAV9 in vivo. Transduction rates in vivo were similar between exo-AAV1 and exo-AAV9, with AAV9 targeting 60% of IHCs and 25% of OHCs after injection (Supplementary FIG. 2b).

### Example 4. Exo-AAV1 Gene Delivery Partially Rescues Hearing in Lhfpl5^{-/-} Mice

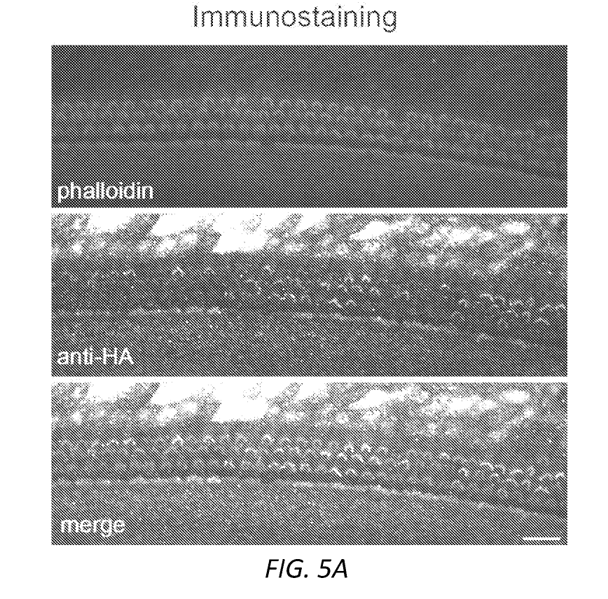

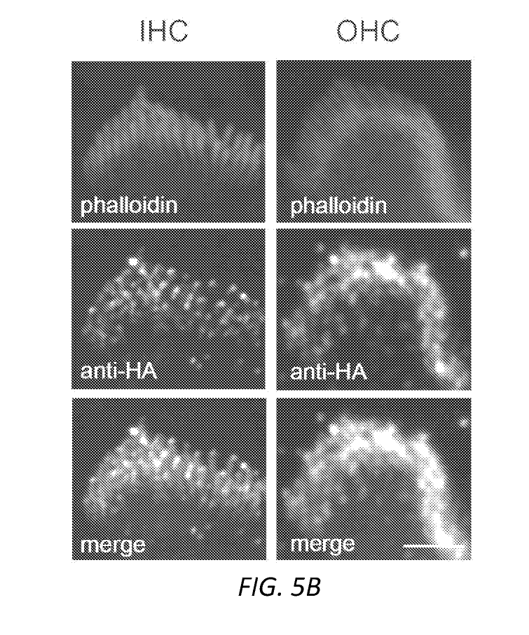

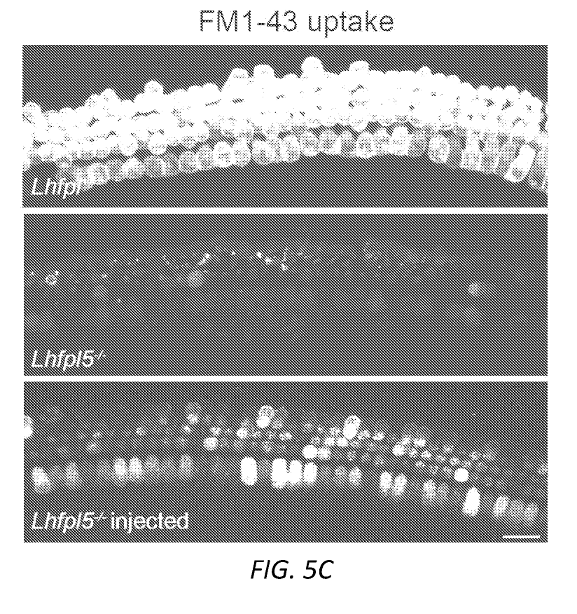

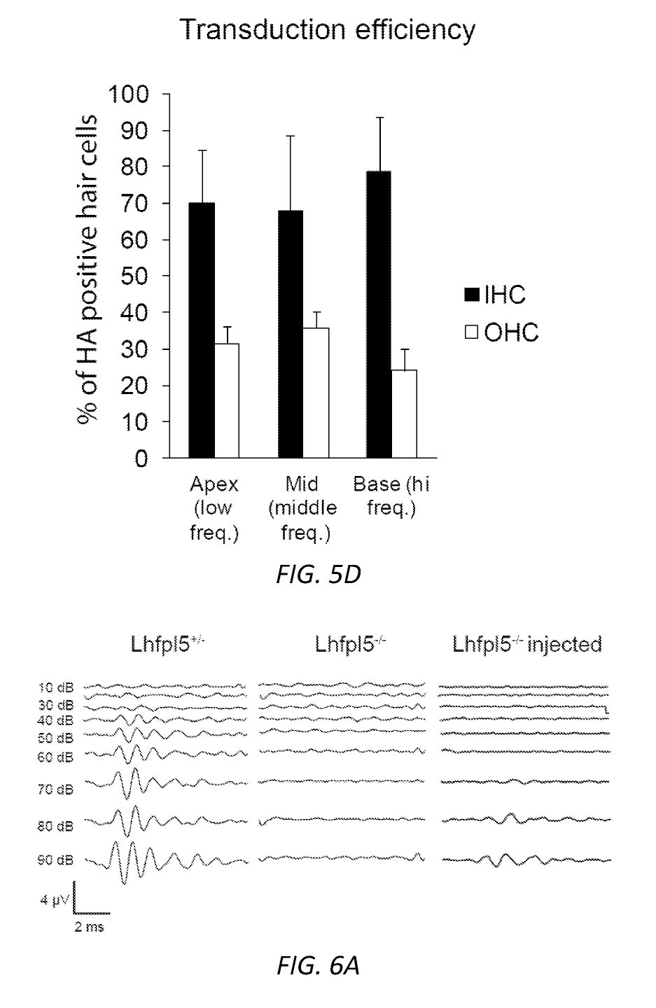

We next tested the ability of exo-AAV to produce efficient expression of a biologically-relevant gene, asking whether exo-AAV1 could improve hearing in a mouse model for human hereditary deafness. We selected a mouse with a targeted deletion of lipoma HMGIC fusion partner-like 5 (Lhfpl5; also known as tetraspan membrane protein of hair cell stereocilia or Tmhs). LHFPL5 protein is an integral component of the mechanotransduction machinery in both OHCs and IHCs and its absence leads to early hair cell degeneration, profound deafness and severe vestibular dysfunction¹⁸. Since this model is on the C57BL/6 background and our previous gene transfer experiments were performed on CD1 mice, we tested whether exo-AAV transduces hair cells with the same efficiency on the C57BL/6 background. We did not observe any differences between CD1 and C57BL/6 transduction rates using exo-AAV1-GFP (Supplementary FIG. 5). Although results were variable, we noted that some of the C57BL/6 animals showed very efficient transduction (>95% IHC and >85% OHC transduction; Supplementary FIG. 6), never achieved with conventional AAV1.

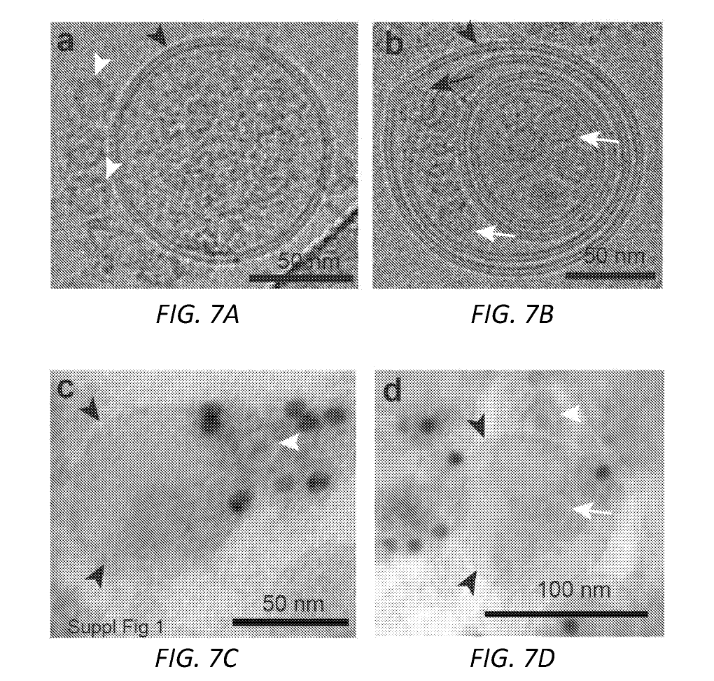

For gene-addition therapy, a mouse codon-optimized gene encoding LHFPL5 with a hemagglutinin (HA) tag at the N-terminus was cloned into an AAV vector backbone under the CBA promoter (Supplementary FIG. 7a). When this exo-AAV1-HA-Lhfpll5 was produced in HEK293T cells, anti-HA immunoblotting of cell lysates revealed bands of the expected molecular weight for LHFPL5 (Supplementary FIG. 7b).

Next, we tested whether this construct restores function in cochlear explant cultures from Lhfpl5^{-/-} animals. Exo-AAV1-HA-Lhfpl5 restored FM1-43 loading in explant cultures (indicating the presence of functional mechanotransduction channels) (FIG. 4a). In addition, anti-HA labeling was present in hair cell stereocilia (FIG. 4b). We quantified average FM1-43 signal in cochlear explants from Lhfpl5^{+/-} mice, Lhfpl5^{-/-} mice, and Lhfpl5^{-/-} mice transduced in culture with two different doses of exo-AAV1-HA-Lhfpl5. The average FM1-43 fluorescence intensity per hair cell in Lhfpl5^{-/-} cochlea was comparable to the background intensity in an area without hair cells. In Lhfpl5^{-/-} cochleas transduced by exo-AAV1-HA-Lhfpl5 vector, FM1-43 intensity was 70% of the Lhfpl5^{+/-} positive control at the highest tested dose (FIG. 4c). FM1-43 intensity increased from apex to base in exo-AAV1-HA-Lhfpl5-treated Lhfpl5^{-/-} cultures, a gradient similar to that seen with the GFP reporter (FIG. 1d). At all points, cellular FM1-43 intensity levels were significantly higher than in untreated Lhfpl5^{-/-} cultures (FIG. 4d). At the base, FM1-43 intensity was as high in exo-AAV1-HA-Lhfpl5-treated Lhfpl5^{-/-} cultures as in heterozygous positive controls (FIG. 4d). These data confirmed that the construct was functional and that the HA tag allowed specific detection of the transgene.

Next, we injected exo-AAV1-HA-Lhfpl5 into the cochlea by RWM injection at P1-2. RWM injection was used rather than cochleostomy, because it was less variable in our hands. Furthermore, we could use a higher volume and therefore dose using RWM injection, and there was less of base-to-apex decrease in transduction with RMW injection compared to cochleostomy (FIG. 2d). For in vivo injection, we administered the maximum injectable volume, based on preliminary experiments: 1200 nl (containing 2.7×10⁹ GCs). Several days later, we dissected cochleas and cultured them 1-2 days before viewing. Anti-HA immunostaining at P4+2 showed distinct signal in stereociliary bundles of both IHCs and OHCs (FIG. 5a). High magnification images revealed anti-HA staining at the tips of stereocilia, including the tallest row, in agreement with the previously reported localization of native LHFPL5¹⁸ (FIG. 5b). We confirmed that exo-AAV-transduced IHCs and OHCs have functional mechanotransduction, as assessed by FM1-43 loading (FIG. 5c). We assessed the efficiency of exo-AAV transduction by counting the hair cells with anti-HA labeling at the bundle and found that 72±17% of IHCs and 30±5% of OHCs exhibited bundle staining, with nearly equal distribution along the cochlea (FIG. 5d).

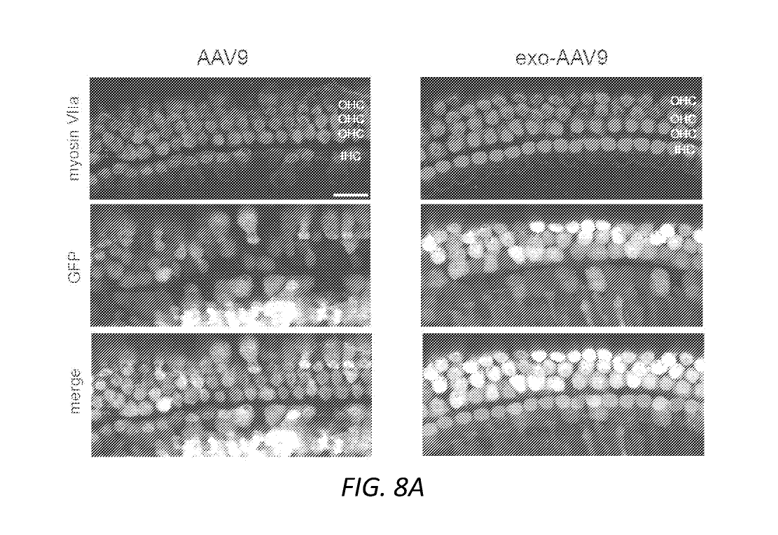

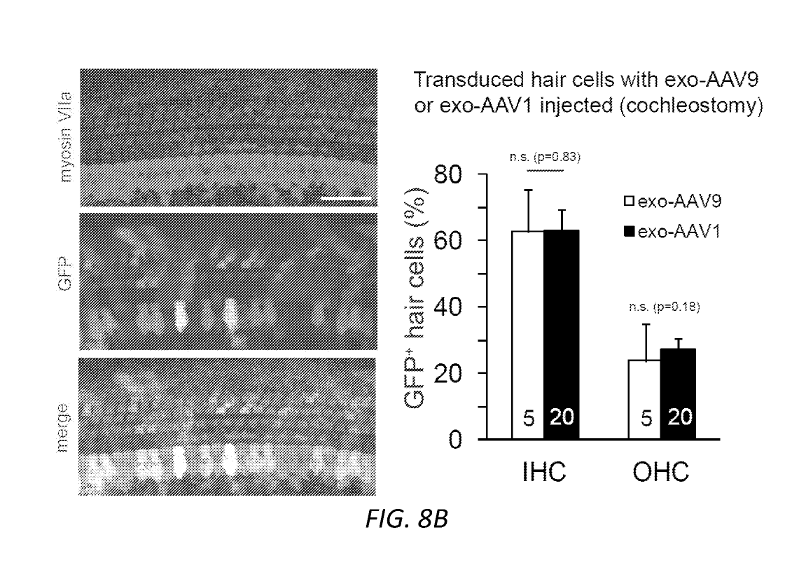

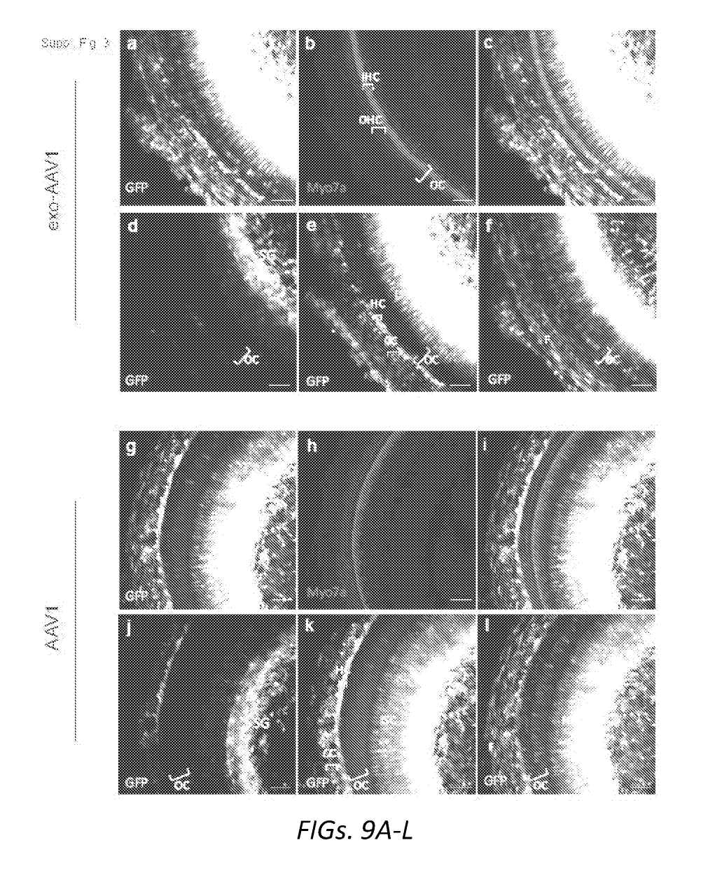

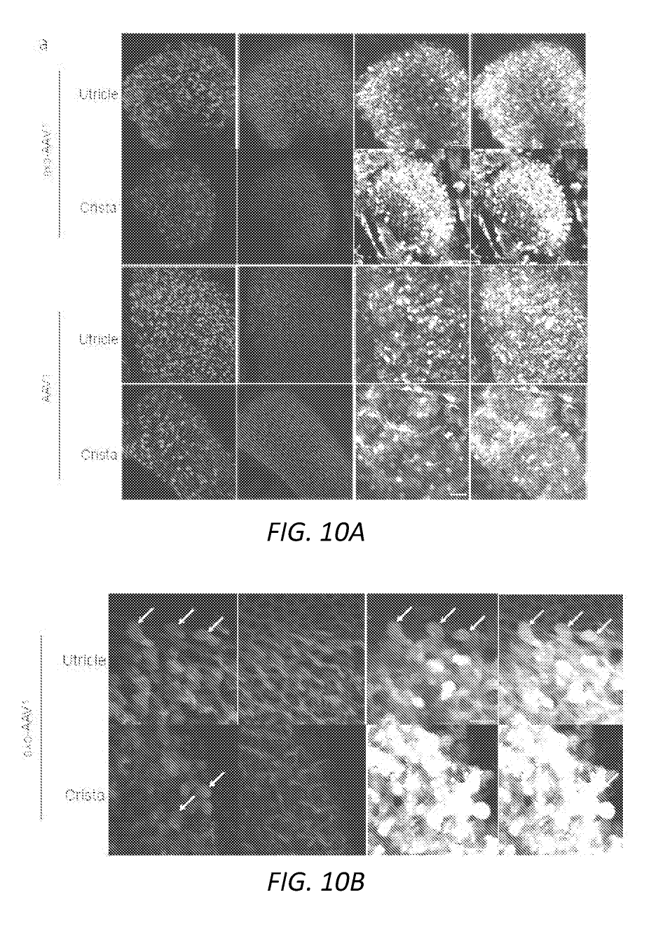

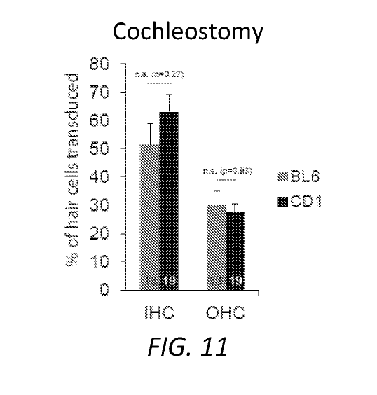

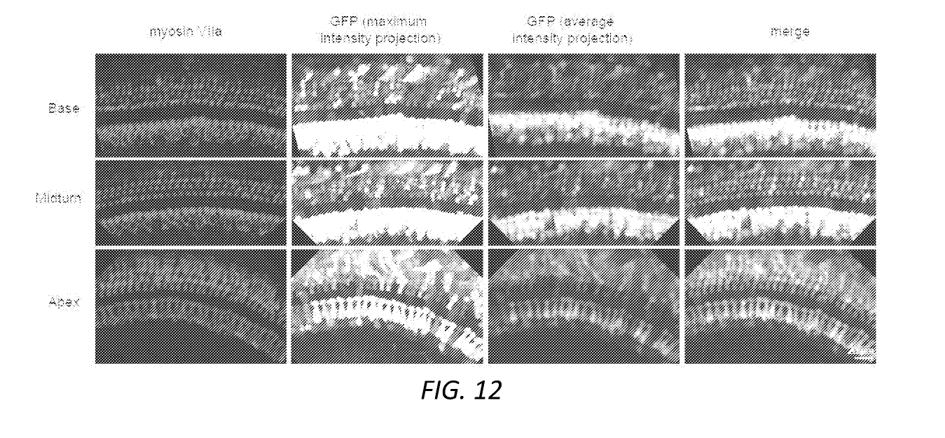

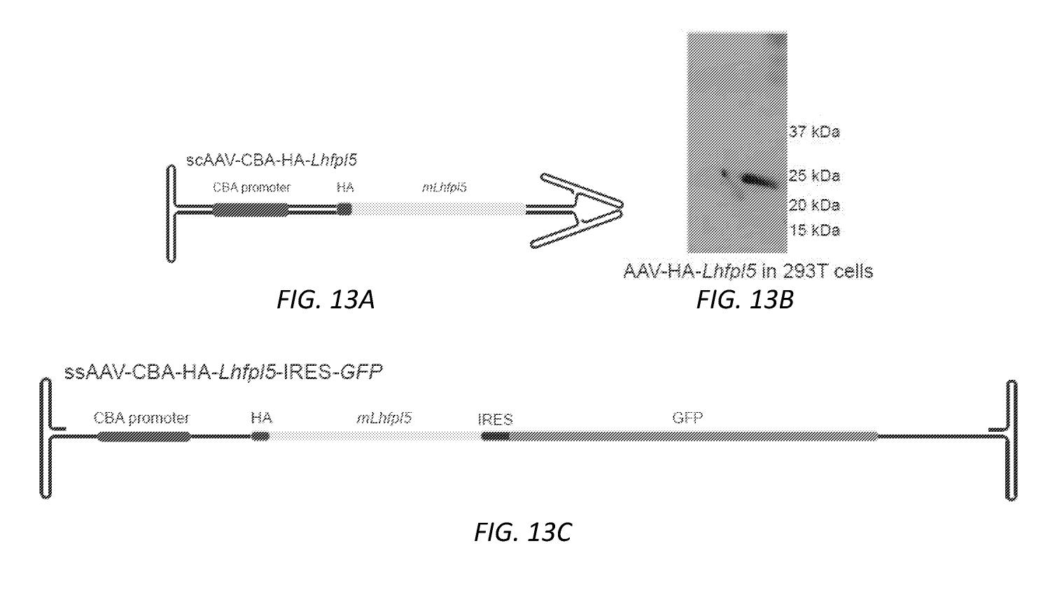

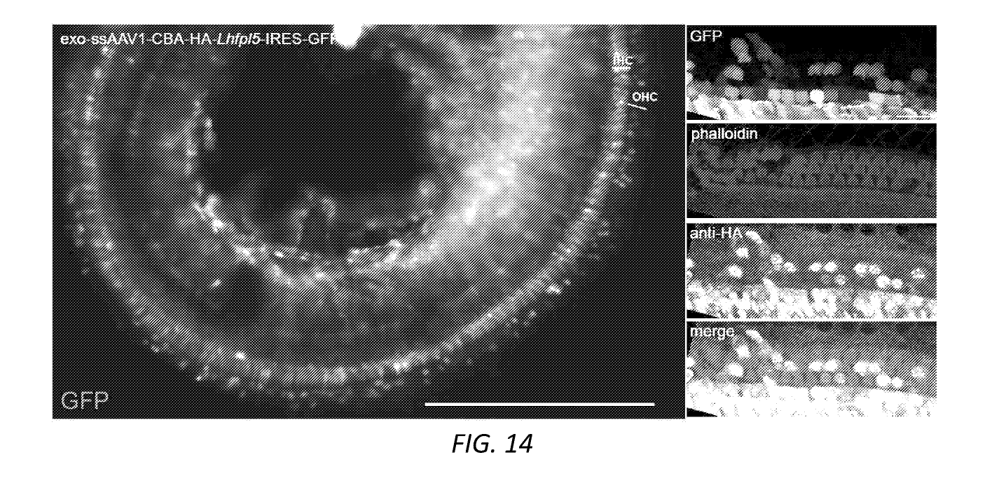

We also tested AAV-HA-Lhfpl5-IRES-GFP packaged in exo-AAV1. This allows co-expression of LHFPL5 and GFP in the same cell. Importantly, all GFP-positive cells exhibited anti-HA staining, confirming specificity of the anti-HA antibody (Supplementary FIG. 8). Some GFP-negative cells also showed anti-HA bundle staining, which may be due to weak translation downstream of the IRES, making GFP undetectable.

To determine whether exo-AAV-mediated gene transfer impairs normal hearing, we tested heterozygous animals injected with exo-AAV1-HA-Lhfpl5 by RWM injection. RWM injection did not alter hearing thresholds, as measured by auditory brainstem evoked responses (ABR)(FIG. 6b), or change ABR P1 or P2 peak amplitudes (FIG. 6c), confirming that both the procedure and the vectors are safe at early ages.

Next, we tested physiological rescue of hearing in deaf mice injected with exo-AAV1-HA-Lhfpl5 and performed ABR recordings at 4 weeks post-injection, using frequencies from 4 to 45 kHz (FIG. 6a-c). Uninjected Lhfpl5^{-/-} animals did not show detectable ABRs at any sound pressure level (SPL) up to 100 dB (FIG. 6a). In Lhfpl5^{-/-} animals injected through the RWM with exo-AAV1-HA-Lhfpl5, we observed improved hearing thresholds at frequencies from 4 to 22 kHz (FIG. 6b) in 9 out of 12 animals. The four animals with the best rescue showed thresholds of ˜70 dB SPL at 8 and 11 kHz, an improvement of ˜30 dB. We never detected ABRs for sound presented to the non-injected side. Although we did not directly analyze gene transfer in the non-injected ear, the ABR data suggest that any gene transfer to the contralateral ear is minimal using this injection protocol. Nevertheless, we have previously reported low levels of gene transfer to inner ear hair cells after intravenous injection of exo-AAV in adult mice, suggesting that it may be possible to transduce both ears under certain conditions¹⁹.

The average peak 2 amplitudes at 90 dB SPL were 0.88±0.18 and 0.84±0.12 V (mean±SEM) at 8 and 11 kHz, respectively, which is approximately 25% of that in normal heterozygotes (FIG. 6c). Latencies of peak 1 and peak 2 were not significantly increased in rescued animals compared to WT animals at the same SPL, except at 11 kHz for peak 1 (FIG. 6c, p<0.01, two tailed t-test). Injected and non-injected heterozygotes did not show a statistically significant difference in the latency of the P1 or P2 ABR peaks (FIG. 6c).

We tested the behavioral correlates of hearing and balance in Lhfpl5 knockouts rescued with exo-AAV. We first found that rescue of hearing by exo-AAV1-HA-Lhfpl5 was sufficient to elicit a startle response to a loud clap, a standard test of hearing (Supplementary Video 1).

Head bobbing and circling are common traits of Lhfpl5 KO mice and may reflect abnormal vestibular function²⁰⁻²². Because GFP-positive hair cells were also evident in vestibular sensory epithelia, suggesting vector diffusion to the vestibular system (FIG. 3), we performed behavioral tests in treated (injected through the RWM with exo-AAV1-HA-Lhfpl5) and nontreated Lhfpl5 KO mice. We performed an open field test, in which animals were placed in a circular arena for 5 minutes. Normal heterozygous mice showed gait and head stability and normal explorative behavior (Supplementary Video 2). On the contrary, Lhfpl5^{-/-} mice exhibited frequent head tossing, gait instability, backward movement and circling. Five out of twelve exo-AAV1-HA-Lhfpl5 treated Lhfpl5^{-/-} animals did not exhibit head tossing, indicating rescue of balance function. Averaging all animals, we found head tossing was significantly decreased in the treated Lhfpl5^{-/-} animals compared to untreated (FIG. 6d) (p<0.001, Mann-Whitney U test). Similarly, circling was analyzed using computerized image analysis. Treated Lhfpl5^{-/-} animals exhibited significantly fewer 360° rotations compared to untreated (FIG. 6d) (p<0.05, Mann-Whitney U test). These results confirm that not only hearing, but the abnormal movements characteristic of compromised balance in these mice are improved after exo-AAV1-HA-Lhfpl5 gene therapy.

### REFERENCES

1. Lin, F. R., J. K. Niparko, and L. Ferrucci, Hearing loss prevalence in the United States. Arch Intern Med, 2011. 171(20): p. 1851-2.
2. Alzheimer's, A., 2016 Alzheimer's disease facts and figures. Alzheimers Dement, 2016. 12(4): p. 459-509.
3. Mason, J. A. and K. R. Herrmann, Universal infant hearing screening by automated auditory brainstem response measurement. Pediatrics, 1998. 101(2): p. 221-8.
4. Geleoc, G. S. and J. R. Holt, Sound strategies for hearing restoration. Science, 2014. 344(6184): p. 1241062.
5. Wise, A. K., T. Tu, P. J. Atkinson, B. O. Flynn, B. E. Sgro, C. Hume, et al., The effect of deafness duration on neurotrophin gene therapy for spiral ganglion neuron protection. Hear Res, 2011. 278(1-2): p. 69-76.
6. Kawamoto, K., S. H. Sha, R. Minoda, M. Izumikawa, H. Kuriyama, J. Schacht, et al., Antioxidant gene therapy can protect hearing and hair cells from ototoxicity. Mol Ther, 2004. 9(2): p. 173-81.
7. Kawamoto, K., M. Yagi, T. Stover, S. Kanzaki, and Y. Raphael, Hearing and hair cells are protected by adenoviral gene therapy with TGF-beta1 and GDNF. Mol Ther, 2003. 7(4): p. 484-92.
8. Li, W., J. Wu, J. Yang, S. Sun, R. Chai, Z. Y. Chen, et al., Notch inhibition induces mitotically generated hair cells in mammalian cochleae via activating the Wnt pathway. Proc Natl Acad Sci USA, 2015. 112(1): p. 166-71.
9. Liberman, M. C., J. Gao, D. Z. He, X. Wu, S. Jia, and J. Zuo, Prestin is required for electromotility of the outer hair cell and for the cochlear amplifier. Nature, 2002. 419(6904): p. 300-4.
10. Kohrman, D. C. and Y. Raphael, Gene therapy for deafness. Gene Ther, 2013. 20(12): p. 1119-23.
11. Sacheli, R., L. Delacroix, P. Vandenackerveken, L. Nguyen, and B. Malgrange, Gene transfer in inner ear cells: a challenging race. Gene Ther, 2013. 20(3): p. 237-47.
12. Akil, O., R. P. Seal, K. Burke, C. Wang, A. Alemi, M. During, et al., Restoration of hearing in the VGLUT3 knockout mouse using virally mediated gene therapy. Neuron, 2012. 75(2): p. 283-93.
13. Askew, C., C. Rochat, B. Pan, Y. Asai, H. Ahmed, E. Child, et al., Tmc gene therapy restores auditory function in deaf mice. Sci Transl Med, 2015. 7(295): p. 295ra108.
14. Fitzpatrick, Z., B. Gyorgy, J. Skog, and C. A. Maguire, Extracellular vesicles as enhancers of virus vector-mediated gene delivery. Hum Gene Ther, 2014. 25(9): p. 785-6.
15. Gyorgy, B., M. E. Hung, X. O. Breakefield, and J. N. Leonard, Therapeutic applications of extracellular vesicles: clinical promise and open questions. Annu Rev Pharmacol Toxicol, 2015. 55: p. 439-64.
16. Gyorgy, B., Z. Fitzpatrick, M. H. Crommentuijn, D. Mu, and C. A. Maguire, Naturally enveloped AAV vectors for shielding neutralizing antibodies and robust gene delivery in vivo. Biomaterials, 2014. 35(26): p. 7598-609.
17. Maguire, C. A., L. Balaj, S. Sivaraman, M. H. Crommentuijn, M. Ericsson, L. Mincheva-Nilsson, et al., Microvesicle-associated AAV vector as a novel gene delivery system. Mol Ther, 2012. 20(5): p. 960-71.
18. Xiong, W., N. Grillet, H. M. Elledge, T. F. Wagner, B. Zhao, K. R. Johnson, et al., TMHS is an integral component of the mechanotransduction machinery of cochlear hair cells. Cell, 2012. 151(6): p. 1283-95.
19. Hudry, E., C. Martin, S. Gandhi, B. Gyorgy, D. I. Scheffer, D. Mu, et al., Exosome-associated AAV vector as a robust and convenient neuroscience tool. Gene Ther, 2016. 23(4): p. 380-92.
20. Gibson, F., J. Walsh, P. Mburu, A. Varela, K. A. Brown, M. Antonio, et al., A type VII myosin encoded by the mouse deafness gene shaker-1. Nature, 1995. 374(6517): p. 62-4.
21. Anderson, D. W., F. J. Probst, I. A. Belyantseva, R. A. Fridell, L. Beyer, D. M. Martin, et al., The motor and tail regions of myosin XV are critical for normal structure and function of auditory and vestibular hair cells. Hum Mol Genet, 2000. 9(12): p. 1729-38.
22. Di Palma, F., R. H. Holme, E. C. Bryda, I. A. Belyantseva, R. Pellegrino, B. Kachar, et al., Mutations in Cdh23, encoding a new type of cadherin, cause stereocilia disorganization in waltzer, the mouse model for Usher syndrome type 1D. Nat Genet, 2001. 27(1): p. 103-7.
23. Yu, Q., Y. Wang, Q. Chang, J. Wang, S. Gong, H. Li, et al., Virally expressed connexin 26 restores gap junction function in the cochlea of conditional Gjb2 knockout mice. Gene Ther, 2014. 21(1): p. 71-80.
24. Longo-Guess, C. M., L. H. Gagnon, S. A. Cook, J. Wu, Q. Y. Zheng, and K. R. Johnson, A missense mutation in the previously undescribed gene Tmhs underlies deafness in hurry-scurry (hscy) mice. Proc Natl Acad Sci USA, 2005. 102(22): p. 7894-9.
25. McCarty, D. M., P. E. Monahan, and R. J. Samulski, Self-complementary recombinant adeno-associated virus (scAAV) vectors promote efficient transduction independently of DNA synthesis. Gene Ther, 2001. 8(16): p. 1248-54.
26. Wang, Z., H. I. Ma, J. Li, L. Sun, J. Zhang, and X. Xiao, Rapid and highly efficient transduction by double-stranded adeno-associated virus vectors in vitro and in vivo. Gene Ther, 2003. 10(26): p. 2105-11.
27. Xiao, X., J. Li, and R. J. Samulski, Production of high-titer recombinant adeno-associated virus vectors in the absence of helper adenovirus. J Virol, 1998. 72(3): p. 2224-32.
28. Rabinowitz, J. E., F. Rolling, C. Li, H. Conrath, W. Xiao, X. Xiao, et al., Cross-packaging of a single adeno-associated virus (AAV) type 2 vector genome into multiple AAV serotypes enables transduction with broad specificity. J Virol, 2002. 76(2): p. 791-801.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of inducing expression of a transgene in a cell of the inner ear, the method comprising delivering to the cell an effective amount of a exosome-associated viral vector comprising the transgene.

2. The method of claim 1, wherein the cell is a hair cell of the cochlea or vestibular system.

3. The method of claim 2, wherein the cell is an inner hair cell of the cochlea or an outer hair cell of the cochlea.

4. The method of claim 3, wherein the subject has a hearing disorder, and the transgene is delivered in a therapeutically effective amount.

5. The method of claim 2, wherein the cell of the vestibular system is a hair cell of the utricle, or a cell in an ampulla of a lateral semicircular canal, or a hair cell in a cupula.

6. The method of claim 2, wherein the cell is a cell of the vestibular system, the subject has a disorder of the vestibular system, and the transgene is delivered in a therapeutically effective amount.

7. The method of claim 1, wherein the transgene is selected from the group consisting of TMIE, TMC1, ATOH1, Lhfpl5, Clrn1, Ush1C (harmonin), PAX3, MITF, EDNRB, EDN3 SOX10, SLC26A4, (Pendrin), KCNA1, CACNA1A, CACNB4, SLC1A3, and CX26/GJB2.

8. The method of claim 1, wherein the exosomes are 50-150 nM in diameter.

9. A composition comprising an exosome-associated viral vector comprising a transgene for use in inducing expression of the transgene in a cell of an inner ear of a subject.

10. The composition for the use of claim 9, wherein the transgene comprises a sequence listed in Table A.


SEQUENCE LISTING (4 sequences)

SEQ ID NO: 1 - DNA, 20 bp, Artificial Sequence
  PCR primer wild type exon 2 forward
tgactgctggatctcagtgc

SEQ ID NO: 2 - DNA, 20 bp, Artificial Sequence
  PCR primer wild type exon 2 reverse
gtttggctgctggtcttagc

SEQ ID NO: 3 - DNA, 20 bp, Artificial Sequence
  PCR primer Lhfpl5 KO forward
tagcaggcatgctggggatg

SEQ ID NO: 4 - DNA, 20 bp, Artificial Sequence
  PCR primer Lfhpl5 KO reverse
tccgctgatggcctttctca